(19) United States
(12) Patent Application Publication
(10) Pub. No.: US 2006/0073173 A1
(43) Pub. Date: Apr. 6, 2006

(54) Large-scale manufacturing process for the production of pharmaceutical compositions

(76) Inventors: Maria Banach, Port Washington, WI (US); Lynn Gold, Seattle, WA (US)

Correspondence Address: WILSON SONSINI GOODRICH & ROSATI, 650 PAGE MILL ROAD, PALO ALTO, CA, 94304-1050, US

(21) Appl. No.: 10/958,510

(22) Filed: Oct. 4, 2004

(51) Int. Cl. A61K 9/00

(52) U.S. Cl. 424/400

(57) ABSTRACT

Methods for manufacturing pharmaceutical compositions with a predetermined surface tension or other properties are provided. Also provided are pharmaceutical compositions with a predetermined surface tension or other properties, articles of manufacture containing the pharmaceutical compositions and systems for preparation of the compositions.

### FIELD OF THE INVENTION

Provided herein are compositions and methods of manufacturing compositions that have a predetermined surface tension.

### BACKGROUND

Assurance of safety and efficacy are important for developing a marketable and useful pharmaceutical composition. Safety and efficacy should be maintained consistently throughout the lifetime of the composition from manufacturing, through storage and use. Consistency can be affected by the properties of the composition and their stability over time and conditions. Properties including concentration of active ingredient, pH, solubility, osmolality, surface tension and purity can affect the efficacy and/or safety of a composition. Such properties are often chosen and optimized using small scale formulation. Compositions can be formulated in single or a small number of doses for experimental testing. Such testing can include measurements of physical and chemical properties and experimental administration in subjects.

Once a formulation is chosen with the desired activity, the formulation can be scaled up for larger volumes and commercial production. Manufacturing of pharmaceutical compositions can include quality control procedures to assure consistency. Properties including identity, strength, quality and purity can be used to monitor compositions during and following manufacture. Quality control steps can be implemented throughout the manufacturing process as well as monitoring the end product. Quality control procedures during manufacture and on the final manufactured composition can assure that the manufactured pharmaceutical composition has the desired identity, strength, quality and purity. Quality control measurements also can compare the compositions measured at large scale to the formulations made at small scale. If the properties of the manufactured composition do not resemble the properties of the small scale formulations, then the earlier efficacy and safety testing done on the small scale formulations may not accurately reflect the same properties in the manufactured compositions. Thus, safety and efficacy of the manufactured composition cannot be assured. Thus, there is a need for methods of manufacture that provide formulation of compositions in large scale that reflect the properties optimized and chosen from small scale formulation and testing.

Therefore, among the objects herein, it is an object to provide methods of manufacture that result in compositions that exhibit consistent properties and to provide such compositions.

### SUMMARY

Provided herein are methods of manufacture that result in compositions that exhibit consistent properties, particularly a predetermined surface tension. Also provided are compositions that exhibit such consistent predetermined properties. Also provided are articles of manufacture containing the compositions in sterile containers packaged for single dosage or multiple dosage administration. The compositions are sterile and contain surfactant near the critical micelle concentration and an agent, such as an antibiotic.

Methods of manufacturing a composition with a predetermined surface tension are provided herein. The methods include formulating a pharmaceutical composition containing an agent at a manufacturing scale. The compositions are manufactured under conditions that reduce or inhibit the formation of non-equilibrium aggregates or other non-equilibrium structure, whereby the resulting composition has a predetermined surface tension. Such compositions also can contain one or more surfactants, at a concentration near the critical micelle concentration (cmc). Such methods can be used to manufacture compositions that contain an agent that has a tendency to form non-equilibrium aggregates such that under the conditions of manufacture the formation of non-equilibrium aggregates or other non-equilibrium structures or intermediates is reduced or inhibited. For example, such that the manufactured composition contains the agent in monomeric form.

The methods of manufacture provided herein also include formulating a pharmaceutical composition at a manufacturing scale that has a predetermined surface tension, where the composition contains an agent and a surfactant. The concentration of surfactant is near the cmc, such that the manufactured composition has a predetermined surface tension. Such methods include concentrations of surfactant below the cmc, about or equal to the cmc and concentrations within 2 fold or 3 fold below or above the cmc. The predetermined surface tension of exemplary manufactured compositions includes a predetermined surface tension of between 30-50 dynes/cm and between 35-45 dynes/cm. The methods of manufacture can include a step of assessing the surface tension of the manufactured composition.

The methods of manufacture herein produce compositions that contain agents selected from among, for example, an anti-infective agent, an anti-inflammatory agent, an antihistamine, an antileukotrienes, a decongestant, an antiviral agent, and an anesthetic and any other suitable active compound. Exemplary anti-infective agents include an aminoglycoside, a macrolide, a penicillin, a quinolone, a cephalosporin, amphotericin B, a polyene, a pyrimidine analog and an azole agent. Exemplary anti-inflammatory agents include a steroid and a non-steroidal anti-inflammatory (NSAID) agent. The manufactured compositions can be formulated for direct administration or for subsequent dilution before administration. The compositions can be unit dose or multidose. The compositions are in liquid form and can be a solution, an emulsion, a suspension or other mixture.

The methods of manufacturing a pharmaceutical composition herein include reducing energy input into the manufacturing process or reducing the free energy of the compositions. The energy input is reduced at a step such as mixing, transferring, filtering and/or filling. In such methods, formation of non-equilibrium aggregates of the agent or other components and of other non-equilibrium structures is reduced or eliminated. In one example, energy input is reduced at a step of filtering by reducing the pressure differential across the filter or reducing the turbulence during filtration. In another example, the methods include a reduced pressure differential during filtration or during filling. The methods of manufacturing a pharmaceutical composition herein also can include filling the composition into containers for storage as multi-dosage or single dosage administration. The filling step is accomplished under aseptic conditions, for example in a closed system. The containers are hermetically sealed.

The methods of manufacture provide compositions with a stable predetermined surface tension. In one example, the predetermined surface tension of the manufactured composition varies less than about 5%, 10%, 20%, 30%, 40% or 50%, generally less than 1% to 10%, over a period of storage time. The period of storage time includes the amount of time between the time of filling storage containers with the manufactured composition and the time of measuring the surface tension of the manufactured composition. Exemplary periods of storage time include 1 hour, 1 day, 1 week, 1 month and 1 year.

The methods of manufacture can be used to formulate manufacturing scale compositions any bio-active agent or other such agent, including those agents described herein. In one example, the methods include manufacturing a pharmaceutical composition that includes tobramycin and a surfactant at a manufacturing scale and that has a predetermined surface tension. The concentration of surfactant is near the cmc and the manufactured composition has a predetermined surface tension between about 30 to about 50 dynes/cm. In one example, the surfactant is polysorbate 20 (Tween-20). The concentration of tobramycin can be chosen for a particular strength and efficacy level dependent on the disease or condition to be treated. In one example, the manufactured compositions include a concentration of tobramycin at or about 5 mg/ml to 100 mg/ml, such as 15 mg/ml, 30 mg/ml or 60 mg/ml.

Provided herein are compositions manufactured using the methods described herein. The compositions are in liquid form and can be aqueous or non-aqueous compositions. In one example, the methods include compositions manufactured at a volume that is at least 1, 2, 3, 4, 5, 10, 15, 20, 25, 50 and 100 liters. In an exemplary method, compositions are manufactured at a volume of at least ten liters. Exemplary manufactured compositions provided herein include a pharmaceutical agent and a surfactant at a concentration near or at the cmc, where the manufactured composition has a predetermined a surface tension; and the volume of the manufactured composition is ten liters or greater. The compositions can include a non-ionic surfactant, for example Tween-20. In one example, the compositions have a predetermined surface tension between 30-50 dynes/cm. In another example, the predetermined surface tension is between 35-45 dynes/cm. The compositions include one or more agents such as an anti-infective agent, an anti-inflammatory agent, an antihistamine, an antileukotrienes, a decongestant, an antiviral agent, and an anesthetic.

Also provided herein are articles of manufacture that contain a pharmaceutical composition containing an agent and a surfactant. The concentration of surfactant near or at the critical micelle concentration. The article of manufacture includes the composition packaged or filled aseptically into a hermetically sealed container. The container can include glass or plastic containers, including vials, ampoules, blisters, syringes and bottles. The container can be a unit dose or multi-dose container. The composition can be for direct administration or dilution. The container is hermetically sealed such as a safety-sealed, heat-sealed or vacuumed sealed container and the resulting article of manufacture is aseptic and hermetically sealed. In one example, the container is filled and sealed using the blow-fill-seal technology. The articles of manufacture provided herein include compositions that have a predetermined surface tension between 30-50 dynes/cm.

The articles of manufacture provided herein include any of the compositions described herein, for example, containing an anti-infective agent, an anti-inflammatory agent, an antihistamine, an antileukotrienes, a decongestant, an antiviral agent, or an anesthetic. Exemplary anti-infective agents include an aminoglycoside, a macrolide, a penicillin, a quinolone, a cephalosporin, amphotericin B, a polyene, a pyrimidine analog and an azole agent. Exemplary anti-inflammatory agents include a steroid and a non-steroidal anti-inflammatory (NSAID) agent. In one example, the article of manufacture contains a composition having an agent effective for the treatment of sinusitis. The articles of manufacture include composition containing a non-ionic surfactant. In one example, the surfactant is not benzalkonium chloride.

Exemplary compositions, articles of manufacture and methods of manufacture of compositions provided herein include, contain and/or formulate a composition of tobramycin having a predetermined surface tension. In one example, the compositions include tobramycin at a concentration of about or at 60 mg/ml to about or at 15 mg/ml, for example of or about 15 mg/ml, 30 mg/ml or 60 mg/ml, and a non-ionic surfactant, for example, polysorbate-20. In one example, the concentration of polysorbate-20 is at or about 0.125 mg/ml or at or about a concentration between 0.009% and 0.015% by weight. The composition also can contain sodium chloride at a concentration between 2.5 mg/ml and 4.3 mg/ml. The composition has a predetermined surface tension between about or at 30 dynes/cm to about or at 50 dynes/cm. Exemplary manufactured compositions include a formulation at a volume greater than about or at ten liters. Exemplary articles of manufacture of tobramycin include single or multi-dose formulations, packaged for direct administration or dilution. The articles of manufacture are packaged into aseptic and hermetically containers.

Also provided herein are systems of manufacture for formulation of a pharmaceutical composition containing an agent and a surfactant, where the amount of surfactant is near the critical micelle concentration. The systems include a filtration apparatus containing a filter, wherein the filter is for sterilization of a pharmaceutical compositions and of configuration that produces low turbulent flow, whereby the resulting composition is produced at a stable predetermined surface tension. In one example, the systems are for formulating compositions at a volume of at least 10 liters. In one example, the system contains a filter that has a pore size and/or distribution that permits or promotes laminar flow. The system also can contain an automated apparatus for aseptically filling and hermetically sealing containers. The system can contain a computer for directing automated operation of the system. The systems can include any of the compositions described herein that contain an agent and a surfactant. In one example, the system includes a composition that contains polysorbate.

Also provided herein are methods of treating chronic sinusitis, comprising nebulizing a composition contained in an article of manufacture. The methods can be used with any of the articles of manufacture described herein. In one example, the article of manufacture contains a composition of an agent effective for treating sinusitis. In another example, the article of manufacture contains a composition of an anti-infective or an anti-inflammatory agent. In an exemplary method, the agent is tobramycin. The compositions can contain a surfactant such as a non-ionic surfactant. In one example of the methods, the composition has a predetermined surface tension between about 30 to about 50 dynes/cm.

### BRIEF DESCRIPTION OF THE DRAWING

FIG. 1 is a block diagram that depicts a typical system of manufacture for large-scale production of the pharmaceutical compositions provided herein and for practicing the methods provided herein. In one embodiment, a computer constructed to provide automated process control of the manufacturing steps is included in the system.

### DETAILED DESCRIPTION

A. DEFINITIONS

Unless defined otherwise, all technical and scientific terms used herein have the same meaning as is commonly understood by one of skill in the art to which the invention(s) belong. All patents, patent applications, published applications and publications, Genbank sequences, websites and other published materials referred to throughout the entire disclosure herein, unless noted otherwise, are incorporated by reference in their entirety. In the event that there are a plurality of definitions for terms herein, those in this section prevail. Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11:942-944.

As used herein, a pharmaceutical composition refers a composition that contains an agent and one or more other ingredients that is formulated for administration to a subject. Pharmaceutical compositions include compositions formulated for single dosage for direct administration as well as multi-dosage compositions and compositions that can be further modified such as by dilution of a concentrate, distribution to another container or other manipulation prior to administration. Pharmaceutical compositions can include additional ingredients including, but not limited to, salts, buffers, surfactants, water, non-aqueous solvents, and flavorings. Pharmaceutical compositions can be aqueous or non-aqueous.

As used herein, an agent refers to an active ingredient of a pharmaceutical composition. Typically active ingredients are active for treatment of a disease or condition. For example, agents that can be included in pharmaceutical compositions include agents for treating chronic sinusitis, such as, but not limited to, anti-infective agents, anti-inflammatory agents, mucolytic agents, antihistamines, antileukotrienes, decongestants, anticholinergics, antifungals and combinations of these classes of agents.

As used herein, a pharmaceutical effect refers to an effect observed upon administration of an agent intended for treatment of a disease or disorder or for amelioration of the symptoms thereof.

As used herein, treatment means any manner in which the symptoms of a condition, disorder or disease or other indication, are ameliorated or otherwise beneficially altered.

As used herein, consistency with reference to properties of a composition, means uniformity and/or unchanging as function of time and under constant conditions. For example, the surface tension of the compositions as manufactured herein are the same or substantially the same (within about 1%, 2%, 5%, 10%, 15%, typically 1-10%) 1 day, 1 week, 6 week, 3 months, 6 months or more after manufacture when assess under the same conditions.

As used herein, a composition refers to any mixture. It can be a solution, a suspension, liquid, powder, a paste, aqueous, non-aqueous or any combination thereof.

As used herein, amelioration of the symptoms of a particular disorder by administration of a particular pharmaceutical composition refers to any lessening, whether permanent or temporary, lasting or transient that can be attributed to or associated with administration of the composition.

As used herein, subject refers to animals, including mammals, such as human beings.

As used herein, a combination refers to any association between or among two or more items. The combination can be two or more separate items, such as two compositions or two collections. It can be a mixture thereof, such as a single mixture of the two or more items, or any variation thereof.

As used herein, a kit refers to a packaged combination. A packaged combination can optionally include a label or labels, instructions and/or reagents for use with the combination.

As used herein, an article of manufacture is a product that is made and sold. For purposes herein, the term refers to containers, such as vials, bottles and ampoules that contain a composition packaged for single or multiple dosage administration and optionally instructions for use thereof.

As used herein, an ingredient of a pharmaceutical composition refers to one or more materials used in the manufacture of a pharmaceutical composition. Ingredient can refer to an active ingredient (an agent) or to other materials in the compositions. Ingredients can include water and other solvents, salts, buffers, surfactants, water, non-aqueous solvents, and flavorings.

As used herein, an intermediate of a pharmaceutical composition refers to any or all intermediate products formed in the formulation of the pharmaceutical composition during a manufacturing process.

As used herein, surfactants refer to a class of molecules that have a hydrophobic tail and a head group that is hydrophilic relative to the tail. Surfactants can be chemical or naturally occurring molecules which, when dissolved in a liquid, for example an aqueous solution, reduce the surface tension. Surfactants also can solubilize non-polar solutes in aqueous solutions. Subclasses of surfactants include anionic, cationic, zwitterionic, gemini and nonionic surfactants.

As used herein, a gemini surfactant refers to a surfactant with two hydrophobic and two hydrophilic groups in the same molecule. Exemplary gemini surfactants include bis(quaternary ammonium halide) geminis such as diethyl ether-α-ω-bis-(dimethylammonium bromide) surfactants.

As used herein, a bile acid is an amphipathic molecule that can form small, water soluble aggregates. Bile acids are amphipathic derivatives of cholesterol. They can be synthesized chemically and enzymatically. Bile acids also can be isolated from synthesis in many organisms including mammals. Bile salts, produced from bile acids, and bile acids tend to self-associate in water with increasing concentration. Aggregates can include dimers and oligomers as well as larger aggregates.

As used herein, aminoglycosides refer to compounds with an amino sugar and amino- or guanido-substituted inositol ring attached by a glycosidic linkage to a hexose nucleus resulting in a polycationic and highly polar compound. Exemplary aminoglycosides include streptomycin, gentamicin, amikacin, kanamycin, tobramycin, netilmicin, neomycin, framycetin, dihydrostreptomycin, dibekacin, streptomycin and paromomycin.

As used herein, penicillin refers to any natural or semisynthetic antibacterial antibiotics derived directly or indirectly from strains of fungi of the genus Penicillium and other soil-inhabiting fungi. Penicillins also include synthetic derived molecules. Exemplary penicilins include, but are not limited to, nafcillin, ticarcillin, clavulanic acid, amoxicillin, ampicillin, bacampicillin, carbenicillin cloxacillin, dicloxacillin, flucloxacillin, methicillin, mezlocillin, oxacillin, penicillin G, penicillin V, piperacillin, pivampicillin, and pivmecillinam. Exemplary penicillins include salts and derivatives of penicillins. Exemplary salts and derivatives include, but are not limited to, aluminum penicillin, benzathine penicillin, benzyl penicillin potassium, benzyl penicillin sodium, clemizole penicillin, dimethoxyphenyl penicillin sodium, penicillin G, penicillin G benzathine, penicillin G potassium, penicillin G procaine, penicillin G sodium, isoxazolyl penicillins, including oxacillin, cloxacillin, and dicloxacillin, penicillin N, penicillin O, penicillin O potassium, penicillin O sodium, phenoxymethyl penicillin, potassium phenoxymethyl penicillin, penicillin V, penicillin V benzathine, and penicillin V potassium.

As used herein, quinolones refers to a class of antibiotic agents. Exemplary quinolones include, but are not limited to, ofloxacin, levofloxacin, ciprofloxacin, norfloxacin, enoxacin, lomefloxacin, grepafloxacin, trovafloxacin, sparfloxacin, temafloxacin, moxifloxacin, gatifloxacin

As used herein, macrolides refers to large ring antibiotic agents. Exemplary macrolide antibiotic agents include, but are not limited to, erythromycin, azithromicin, clindamycin, and clarithromycin.

As used herein, cephalorsporins refers to any natural or semi-synthetic antibacterial antibiotic derived directly or indirectly from strains of Celphalosporium acremonium fungi. Exemplary cephalosporins include, but are not limited to, cefuroxime, ceftazidime, ceftriaxone, cefotaxime, cefoxitin, cefazolin, cephalexin, cephradine, cefadroxil, loracarbef, cefprozil, cefaclor, cefotetan, cefamandole, cefixime, cefpodoxime, and cefizoxime.

As used herein, an anti-inflammatory agent refers to steroidal and non-steroidal anti-inflammatory agents. Exemplary steroidal anti-inflammatory agents include, but are not limited to, betamethasone, fluticasone, mometasone, triamcinolone, dexamethasone, prednisone, beclomethasone, flunisolide, and budesonide. Exemplary non-steroidal anti-inflammatory agents include, but are not limited to, fenoprofen, flurbiprofen, ibuprofen, ketoprofen, naproxen, oxaprozin, diclofenac, etodolac, indomethacin, ketorolac, nabumetone, sulindac tolmetin meclofenamate, mefenamic acid, piroxicam and suprofen.

As used herein, an antifungal agent refers to an agent that inhibits or prevents fungal infection or fungal growth. Antifungal agents include, but are not limited to, exemplary non-azole agents amphotericin B, griseofulvin, haloprogin, and tolnaftate, polyenes such as nystatin, allylamines such as naftifine and terbinafine, pyrimidine analogs, such as flucytosine (5-flurocytosine). Exemplary azole antifungal agents include, but are not limited to, butoconazole, clotrimazole, econazole, fluconazole, itraconazole, ketoconazole, miconazole, oxiconazole, sulconazole and terconazole.

As used herein, a ring structure with reference to a structure of a molecule can include rings that include at least one carbon atom and one or more atoms such as nitrogen, sulfur and oxygen. Ring structures include single rings, and two or more rings. Ring structures can include fused rings.

As used herein, a micelle refers to an association of molecules whereby the molecules are associated with hydrophobic groups oriented towards the interior of the association and polar groups oriented outward towards the solution. Micelles can include associations of a single type of molecule. Micelles also include an association of a mixture of molecules, also referred to as mixed micelles. Micelles can vary in shape including discoidal, globular, spherical, and elliptical micelles.

As used herein, a pre-micelle aggregate refers to a small aggregate formed in the transition between the monomeric state of a molecule and micelles formed from that molecule. Pre-micellar aggregates can include dimers, vesicles and lamellae.

As used herein, a non-equilibrium aggregate is any association of two or more molecules where the association is not an equilibrium structure. Non-equilibrium aggregates include micelles with a diameter less than the diameter of stable equilibrium micelles and pre-micellar aggregates.

As used herein, a molecule that has a tendency to form non-equilibrium aggregates refers to molecules that form non-equilibrium aggregates under particular conditions of formulation and manufacture. For example, conditions that include increased energy input and turbulence can increase the formation of non-equilibrium aggregates. Structural characteristics of molecules including, but not limited to, ring structures, rings with nitrogens, double bonded oxygens, and side groups available for hydrogen bonding can contribute to the tendency of a molecule to form non-equilibrium aggregates.

As used herein, a vesicle refers to a bilayer structure with closed edges that is formed from the association of two or more molecules. In a vesicle, the hydrophobic interior of the bilayer is not exposed to the surrounding solution. For example, two molecules of a gemini surfactant can associate with their hydrophilic groups at opposite ends of the structure and their hydrophobic groups oriented towards each other in a bilayer.

As used herein, critical micelle concentration (cmc) is the approximate concentration at which individual monomers (and in some cases pre-micelle aggregates) associate to form micelles. For example, for many surfactants, at concentrations above the cmc, surfactant compositions contain micelles and monomers of surfactant in equilibrium.

As used herein, near the cmc refers to a concentration of surfactant, that is titrated such that the concentration is sufficient to achieve the predetermined surface tension in a particular composition and the concentration is within or near the range of concentration where micelles are in equilibrium with monomers present in the solution. Generally, near the cmc refers to concentrations equal to the cmc, just above the cmc or just below the cmc. Concentrations near to the cmc include concentrations within 2 times and 3 times the cmc. For example, if the cmc for a surfactant is 0.06 mM, near the cmc generally includes 0.01 mM, 0.02 mM, 0.03 mM, 0.04 mM, 0.05 mM, 0.06 mM, 0.07 mM, 0.08 mM, 0.09 mM, 0.10 mM, 0.11 mM, 0.12 mM, 0.13 mM, 0.14 mM, 0.15 mM, 0.16 mM, 0.17 mM, 0.18 mM and any concentrations in between. The concentration of surfactant that is “near the cmc” is dependent on the nature of the composition since the cmc of a surfactant is affected by temperature and other agents, such as salts, other surfactants, and surfactant-like agents. “Near the cmc” can be empirically determined, calculated or assessed based on known values. In practice, the cmc can be determined for example, by titrating a surfactant in a composition and measuring surface tension. The concentration of surfactant where the surface tension reaches a plateau is designated the cmc of the surfactant in that composition.

As used herein, surface tension plateau with respect to the relation of surface tension to concentration of a surfactant refers to the point at which increasing the concentration of surfactant does not decrease the surface tension of the solution. At concentrations of surfactant below the plateau point, addition of surfactant decreases surface tension in proportion to the concentration of surfactant. At the plateau, addition of surfactant no longer measurably affects the surface tension of the solution.

As used herein, low concentrations of surfactant refer to surfactant concentrations that are titrated to reach a desired predetermined surface tension and reduce or avoid unwanted side effects such as dryness and irritation upon administration of the composition to a subject. Generally, compositions containing a low concentration of surfactant include titrating a surfactant such that the concentration is as little as possible to achieve a predetermined surface tension. Generally, low concentrations of surfactant are below the concentrations typically used for solubilization of an agent. Typical concentrations for solubilization are between 1%-20% by weight. Generally, low concentrations of surfactant are concentrations near the cmc. Also generally, low surfactant concentrations are below 1% by weight of the composition. For instance, typical low concentrations of surfactant include concentrations of 0.9%, 0.8%, 0.6%, 0.5%, 0.4%, 0.3%, 0.2%, 0.1%, 0.08%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001% and less than 0.001% by weight. The amount of surfactant will depend on the types and species of surfactant, the other ingredients and agent(s) in the composition, the mode of administration and the desired predetermined surface tension. The low concentration of surfactant can be determined empirically, calculated, assessed or known using methods and knowledge in the art.

As used herein, surface tension refers to a measurement of the tension acting at an interface of a liquid, such as at the interface of a liquid and air. Surface tension is referred to herein in units of dynes/cm and mN/m, such units of measurement for purposes herein are equivalent.

As used herein, a predetermined surface tension refers to a designated or selected surface tension of a composition. A surface tension can be selected based upon an intended use for a composition. For example, a predetermined surface tension can be chosen such that a composition with such surface tension is effective for retention of an agent in the nasal sinuses. A predetermined surface tension can be selected empirically, such as by determining a surface tension or surface tension range that confers a desired functionality or efficacy to the composition. A predetermined surface tension can be selected from small-scale formulation of the composition. The selected surface tension can then be designated as a predetermined surface tension at which to formulate a manufactured composition, such as in a larger scale process. A predetermined surface tension can include a range of surface tension, for example a predetermined surface tension can be about 30 dynes/cm to about 50 dynes/cm. A predetermined surface tension can be designated as a surface tension that results immediately following manufacture, as stored and/or as administered to a subject. A stable predetermined surface tension refers to a designated surface tension that is attained immediately following manufacture and maintained subsequent to manufacture when stored under suitable conditions, for example, stored under constant temperature, pressure, and humidity.

As used herein, a liquid form refers to a composition in any type of liquid form known in the art. Such forms include, but are not limited to, solutions, emulsions and suspensions. Liquid forms include aqueous and non-aqueous liquids.

As used herein, energy input refers to any type of energy brought to bear on a process such as a manufacturing process. Energy inputs include mixing and agitation, including speed and force of mixing, thermal energy, and pressure.

As used herein, a dimer refers to an association of two molecules. Dimers include homodimers, associations of two of the same molecules, and heterodimers, association of two different molecules.

As used herein, an aggregate refers to an association of two or more molecules. Aggregates include, but are not limited to micelles, vesicles, dimers and other oligomers.

As used herein, a manufacturing process refers to the steps of a method used to prepare a composition in a manufacturing batch amount. A manufacturing process can include, but is not limited to, steps of mixing, limiting, filtering and transferring one or more materials of a composition, including filling. Generally, one or more steps of a manufacturing process are performed by mechanical means such that the energy or force applied is supplied in a mechanized form.

As used herein, preparation at manufacturing scale amounts refers to the preparation of pharmaceutical compositions in amounts greater than single use or single prescription and typically in amounts of at least 5 liters, 10 liters, 20 liters, 25 liters or more. Preparation of manufacturing scale amounts includes preparations intended for clinical or commercial use. Such compositions are manufactured with current Good Manufacturing Practices (see for example, cGMP; 21C.F.R. §§ 210 and 211, Guidance for Industry Sterile Drug Products Produced by Aseptic Processing: Current Good Manufacturing Practice, DRAFT GUIDANCE, (August, 2003), U.S. Department of Health and Human Services, Food and Drug Administration, Center for Drug Evaluation and Research (CDER), Center for Biologics Evaluation and Research (CBER), Office of Regulatory Affairs (ORA) and Guideline on Sterile Drug Products Produced By Aseptic Processing, (June, 1987), Center for Drug Evaluation and Research (CDER), Center for Biologics Evaluation and Research (CBER), Office of Regulatory Affairs (ORA), FDA).

Exemplary manufacturing scale amounts include preparations of pharmaceutical compositions in volumes of one liter or greater. For example, preparation of a manufacturing scale amount of a pharmaceutical composition can include preparation of 1, 2, 3, 4, 5, 10, 15, 25, 50, 100, 500, 1000 and more liters. Typically, manufacturing scale refers to volumes of ten liters or greater. Amounts for manufacture can be based on factors including, but not limited to, dosage, volume/weight, cost, equipment capacity, process limitations, and marketing projections. Typically, compositions that are not manufactured in manufacturing scale amounts refer to methods of making compositions for experimental use in small scale, or at compounding pharmacies and are typically less than 1 liter, 2 liters, 3 liter, 4 liters or 5 liters, depending upon the dosage. Generally many of the steps are not automated but are manually performed.

As used herein, filling refers to the step or steps in a manufacturing process of transferring a composition into one or more storage containers. Such containers can include single and multi-dose vials. Filling also can include transferring into one or more containers for intermediate (temporary) storage that is followed by another transferring step to storage containers. An exemplary filling procedure is the blow-fill-seal method (see for example, Business Briefing Pharmagenerics 2002).

As used herein, a manufactured composition refers to a composition following the manufacturing process, i.e. the end product of the manufacturing process. Manufactured compositions include compositions immediately following manufacture and after a period of storage.

As used herein, storage refers to a period of time following manufacture. Storage can include periods of time, including but not limited to, one or more days, one or more weeks, one or more months, and one or more years.

As used herein, the Reynolds number or N_{R} refers to a dimensionless quantity used to determine whether the flow of a fluid is turbulent or laminar (Sternheim and Kane, General Physics (John Wiley & Sons, 1986), page 300). N_{R} is defined by the equation:

$${N}_{R} = \frac{ρ vD}{η}$$

where:
ρ=density (in kg/m³)
D=diameter (m)
v=average velocity of fluid (m/sec)
η=absolute viscosity (Pa·s)

As used herein, capacity of a filter refers to the volume of fluid that can be processed at a maximum allowable pressure differential and is generally expressed in liters per square meter (L/m²) of filter frontal area.

As used herein, the characteristic flow length refers to the distance a liquid flows in a membrane.

Laminar flow, as used herein, refers to the flow of a fluid in flow lines or streamlines where there is very little mixing between the flow lines. Laminar flow is generally defined as having a Reynolds number, N_{R}, of less than 2,000. The energy required to increase the average fluid velocity of a fluid in laminar flow increases linearly with the average fluid velocity.

Turbulent flow, as used herein, refers to a random flow of a fluid, where the fluid flow lines break down with the formation of eddies. The eddies form in a random manner and will break down to form smaller eddies. The velocity of fluids in turbulent flow continuously change leading to increased shear of the fluid. Turbulent flow is generally defined as having a Reynolds number, N_{R}, of greater than 3,000. In a pipe flow system, the energy required to increase the average fluid velocity of a fluid in turbulent flow increases almost exponentially with the average fluid velocity.

Transient flow or transition flow as used herein refers to a fluid having a Reynolds number, N_{R}, of between 2,000-3,000. Transient or transition flow is unstable, and occurs between laminar and turbulent flow. Flow lines will break down and then form again. The flow pattern thus can fluctuate between laminar and turbulent flow.

As used herein, pressure differential or pressure drop refers to the difference in magnitude between some pressure value and some reference pressure.

As used herein, pressure in a fluid is defined as the normal force per unit area exerted on a imaginary or real plane surface in a fluid. The equation for pressure can expressed as:
p=F/A
where
   p=pressure [lb/in² (psi) or lb/ft² (psf), N/m² or kg/ms² (Pa)]
   F=force [lb (or pound force), N]
   A=area [in² or ft², m²]

As used herein, absolute pressure is measured relative to the absolute zero pressure—the pressure that would occur at absolute vacuum.

As used herein, gauge pressure refers to the pressure read on a pressure gauge. Gauge pressure measures only the amount of pressure above (or below) the atmospheric pressure and is often used to measure the pressure difference between a system and the surrounding atmosphere. Pressure values are generally given in gauge pressures and are designated as psig. Gauge pressure can be expressed as:
p_{g}=pₐ−pₒ
where
   p_{g}=gauge pressure
   pₒ=atmospheric pressure
   pₐ=absolute pressure measured relative to the absolute zero pressure.

As used herein, atmospheric pressure refers to the pressure in the surrounding air. It varies with temperature and altitude above sea level. In imperial units the Standard Atmospheric Pressure is 14.696 psi.

As used herein, flux rate refers to the flow rate of a fluid per unit cross-sectional area.

As used herein, flow rate refers to the volume of fluid that flows past a point per unit time.

As used herein, membrane refers to a thin barrier in which the separation of components occurs on the basis of a different permeability of components (affected by various driving forces) through this barrier. The material of a membrane influences the practicability of the process or the efficiency of separation. A number of materials can be used for membrane construction, including, but not limited to, polymeric materials such as cellulose derivatives, such as cellulose nitrate and cellulose acetate, acrylates, polyamides, polysulphones, polyvinyl chloride, polyvinylidene difluoride (PVDF), polypropylene, polycarbonate, PTFE, fluorinated plastic, sintered metal, metal oxides, sintered glass, carbon, zirconia coated carbon and ceramics, for example those prepared on the basis of Al₂O₃, ZrO₂ or TiO₂. Separation abilities of a membrane are characterized by their cut-off or separation power. Membranes can be produced as sheets or tubes of varying diameter, including capillary tubes with an internal diameter ranging from about 4 mm to about 20 mm, and hollow fibers with internal diameters of less than 1.5 mm. The membranes can be provided in protective casings or modules, and the membrane modules can be of a plate, spiral wound, tubular, fiber bundle or capillary design.

Separation power or cut-off, as used herein, refers to the size (diameter) of particles that is prevented from passing through a membrane or retained on or within the membrane. In the case of microfiltration membranes, cut-off is usually expressed by a mean size of pores (usually given in μm).

As used herein, incompressible fluid refers to a fluid having a pressure differential at equal flow rates independent of the absolute pressure of the system.

As used herein, rheology refers to a study of the change in form and flow of matter under the influence of stresses, embracing elasticity, viscosity, and plasticity. For example, when liquids are subjected to stress they will deform irreversibly and flow. The measurement of this flow is the measurement of viscosity.

As used herein, shear rate refers to shearing forces a liquid experiences. Its unit of measure is called the “reciprocal second” (sec⁻¹).

As used herein, shear stress refers to the force per unit area required to produce the shearing action. Its unit of measurement is “dynes per square centimeter” (dynes/cm²).

As used herein, viscosity refers to the tendency of a fluid to resist flow and is defined as shear stress divided by shear strain. The fundamental unit of viscosity measurement is the “poise.” A material requiring a shear stress of one dyne per square centimeter to produce a shear rate of one reciprocal second has a viscosity of one poise, or 100 centipoise. Viscosity measurements also can be expressed in “Pascal-seconds” (Pa·s) or “milli-Pascal-seconds” (mPa·s), which are units of the International System and are sometimes used in preference to the Metric designations. One Pascal-second is equal to ten poise; one milli-Pascal-second is equal to one centipoise. To be correct, the conditions used to measure the viscosity must be given. This is due to the fact that non-ideal liquids have different values of viscosity for different test conditions of shear rate, shear stress and temperature.

As used herein, a Newtonian fluid or a fluid that has a Newtonian flow is a fluid whose viscosity is independent of the shear on the fluid. Examples of Newtonian liquids are mineral oil, water and molasses.

As used herein, a pseudoplastic fluid is a liquid having a viscosity that changes with the shear it encounters, and specifically for a fluid where increasing shear rate results in a gradual decreasing shear stress, or a thinning of viscosity with increasing shear.

B. Compositions

Provided herein are pharmaceutical compositions that have a predetermined surface tension. The predetermined surface tension of such compositions contributes to the efficacy, stability and utility of the composition. Included in the compositions provided herein are pharmaceutical compositions that have a predetermined surface tension for retention, penetration and/or deposition of an agent in the nasal sinuses.

Also provided herein are methods for manufacturing compositions having a predetermined surface tension, such that the surface tension is stably maintained in the manufactured composition. It is desirable that the surface tension of manufactured pharmaceutical compositions be relatively stable to the manufacturing process. It also is desirable that compositions tested for quality control at the end of the manufacturing process are representative of compositions that are packaged and stored, as well as representative of the composition used for administration and treatments. If composition properties are not stable, quality control measurements made on such compositions after manufacture may not reflect the compositions used in treatment. Such instability can prevent or interfere with regulatory approvals of the compositions. The effectiveness of such compositions for treatments of diseases and conditions can vary if a predetermined surface tension is not stably maintained.

1. Preparation of Compositions with a Predetermined Surface Tension

The surface tension of a composition at equilibrium is stable under constant conditions (e.g. constant temperature, pressure). When a composition is not at equilibrium, properties of the composition such as surface tension can change over time until the composition reaches equilibrium. Thus, preparation of a composition having a stable predetermined surface tension can be achieved by formulating a composition at equilibrium and manufacturing the composition such that it remains at equilibrium at the conclusion of the manufacturing process.

Interactions between the conditions during manufacture and the ingredients of the pharmaceutical composition can effect whether an equilibrium or non-equilibrium state is achieved during manufacture. The methods herein include conditions that can be used to prepare compositions in equilibrium such that the composition has a stable predetermined surface tension. The manufacturing methods herein include the formulation of pharmaceutical agents that contribute to a non-equilibrium state under particular conditions of manufacturing. In one aspect of the methods, pharmaceutical compositions contain an agent that has a tendency to form non-equilibrium aggregates are manufactured under conditions that reduce or eliminate non-equilibrium aggregate formation, such that a predetermined surface tension is attained and maintained in such manufactured compositions.

2. Aggregation Effects on Surface Tension

Non-equilibrium aggregates are intermediate structures. Over time non-equilibrium structures change in size and/or form until equilibrium structures are formed. For example, non-equilibrium aggregates can disperse into monomers over time, where the monomers are a stable equilibrium structure. Alternatively, non-equilibrium structures can grow into larger, more stable aggregates. For example, surfactants form stable micelles as the concentration of surfactant is increased; micelles of a certain size range and containing a certain number of molecules are stable equilibrium structures.

Non-equilibrium aggregates can include dimers, trimers, and small oligomers as well as larger structures. Aggregates that are non-equilibrium structures can be determined empirically and distinguished from equilibrium structures. For example, aggregation state can be measured over time. Aggregates at equilibrium remain in the same aggregation state, whereas non-equilibrium aggregates change size and/or form over time. Aggregation state can be measured by static or dynamic light scattering measurements, NMR, or any other means known in the art for measuring aggregation state of a molecule. Surface tension measurements also are indicative of aggregation state. Surface tension measurements taken over a series of time points can indicate definitive abrupt changes in surface tension as one structure becomes predominant within a solution.

Aggregates that are not true equilibrium structures (non-equilibrium aggregates) can contribute to the instability and variation of the surface tension of a composition. For example, a solution containing non-equilibrium aggregate structures can have a surface tension that changes over time until equilibrium is reached.

In some cases, non-equilibrium aggregate formation is increased by the methods of formulation. For example, formulation under manufacturing conditions, in large volumes and with mechanical, non-manual processes, can increase the amount of non-equilibrium aggregates formed in the composition. Formation of non-equilibrium aggregates can contribute to the instability of surface tension of the formulated compositions. Consequently, compositions with ingredients and agents that have a tendency to form non-equilibrium aggregates can be difficult to manufacture with a predetermined surface tension. The surface tension of such compositions can vary during and/or subsequent to the manufacturing process. Instability of surface tension can be the result of direct and indirect effects of aggregation. Direct effects include agents and/or ingredients with surface-active properties that interact at the air-water interface of the solution. Indirect effects include effects on micelle size and size distribution of micelles in a solution.

One example of compositions that can exhibit instability of surface tension properties includes compositions containing an agent and/or an ingredient that has surface-active properties. The formation of aggregates can alter the surface tension of the composition by removing the surface-active agent or ingredient from the interfacial surface (e.g. the water-air interface), thus resulting in a higher-than expected surface tension. Subsequent breakdown of the aggregates and release of monomers allows the surface-active ingredient to interact at the surface of the solution, thereby reducing the surface tension of the composition. If the formation of such aggregates and transition between aggregates and monomers is not an equilibrium process, the surface tension will fluctuate as the aggregates disperse into monomers over time.

Aggregates of an agent and/or ingredient also can have an indirect effect on surface tension. For example, molecules of an agent or ingredient can form pre-micelle aggregates or small unstable vesicular micelles. The pre-micelle aggregates/vesicles that form are not thermodynamically stable structures. Over time, the agents and ingredients within the aggregates may either partition into larger, stable micelles with a thermodynamically favorable distribution of molecules within the final structure or they may disperse as monomers into the surrounding solution, thus effecting a change in the surface tension. The nature of the agent and other ingredients of the solution determine which route occurs. For example, smaller micelles can increase the polarity and the surface tension of the solution; upon interacting to form larger micelles and reducing the surface charge of the micelle, the surface tension decreases.

3. Model Molecules that Affect Surface Tension

The formation of non-equilibrium aggregates and their effects on surface tension can be influenced by the structure and properties of the aggregating molecule. Surfactants are examples of surface-active agents that participate in equilibrium and non-equilibrium aggregate formation. Surfactants can form large aggregates known as micelles where the molecules of surfactant are oriented such that the hydrophobic portion of the molecule points towards the interior of the micelle and the hydrophilic portion of the molecules point towards the exterior of the micelle. In general, micelles are equilibrium structures, such that above the critical micelle concentration (cmc), micelle formation is in equilibrium with monomers present in the solution. The monomer concentration in solution reaches a maximum; the micelles have a much higher solubility so as the surfactant concentration increases, the free monomer concentration remains constant but the micelle concentration increases.

Some surfactants, however, are capable of forming pre-micellar aggregates. Pre-micellar aggregates are smaller aggregate structures which at higher concentrations of surfactant are converted to micelles. One such example is formation of pre-micellar aggregates by cationic gemini surfactants. These surfactants have two hydrophobic and two hydrophilic groups in the molecule. Two molecules of cationic gemini surfactant can associate with their hydrophilic groups at opposite ends of the structure and their hydrophobic groups oriented towards each other in a bilayer. These structures are not true equilibrium structures. Thermodynamic equilibrium is reached when the structures disperse into monomers over time.

Gemini surfactants, like other cationic surfactants, are effective at reducing surface tension of a solution. The surface-active molecules migrate to the solution interface and form a monolayer in which the hydrophobic portion is oriented to the air and the hydrophilic moieties remain in the aqueous solution. Only surfactant monomers participate in the monolayer. Pre-micellar aggregates do not participate in reducing surface tension. Therefore such surfactants are most effective at reducing surface tension at or near the cmc. Additionally, the formation of pre-micellar aggregates can alter the surface tension properties of the solution, usually by increasing the surface tension.

Other molecules form aggregates in a different manner from surfactants. For example, planar molecules including dyes, nucleic acids and compounds with aromatic or heteroaromatic structures can form open-ended, continuous and step-wise patterns of aggregation. The aggregates are formed through a stacking of the molecules in either back-to-back or front-to-back orientations. In many of these molecule aggregates each face of the molecule has a similar hydrophobicity. In some cases, however, molecules, including bile acids, have faces that are not equal in hydrophobicity.

Bile acid salts and other steroidal structures are rigid planar molecules. The molecules can self-associate and interact with other molecules in a solution. They also can exhibit stacking behavior. For example, bile acids can be used in compositions to solubilize another less soluble molecule. Bile acid salts can sandwich a less soluble molecule, with the hydrophobic face of the bile acids oriented to the less soluble molecule and the hydrophilic face of the bile acid remaining exposed to the aqueous solution.

Bile acid salts also can self-aggregate. Their rigid structure contains a hydrophilic and a hydrophobic face. In aggregates, the hydrophobic face of the bile acid is oriented towards the other molecule(s) in the aggregate; the hydrophilic face interacts with the aqueous environment. Unlike many other detergents, such as anionic detergents, bile acid salts aggregate over a wide concentration range. Such aggregation is concentration dependent. At lower concentrations bile acids form small aggregates such as dimers and oligomers. As the concentration is increased, larger aggregates are formed. The surface tension of a solution containing a bile acid salt can depend on the aggregate state of the molecule.

4. Pharmaceutical Agents that Form Non-Equilibrium Aggregates

In addition to surfactants and bile acids, other classes of molecules can form non-equilibrium aggregates. Provided herein are pharmaceutical agents that have properties similar to gemini surfactants and to bile salts such that they form non-equilibrium aggregates, such as pre-micellar aggregates. Such agents can alter the surface tension of compositions because they form non-equilibrium structures. Over time, for example, the aggregates partition into stable micelles or disperse to monomers and the surface tension of the solution is altered as a result.

The compositions herein include agents that have a tendency to form non-equilibrium aggregates when formulated in a liquid composition. In one example, the compositions are aqueous and contain an agent that has a tendency to forms non-equilibrium structures in water (or an aqueous composition). For example, an agent has hydrophobic groups that orient towards each other in a non-equilibrium aggregate. Such an agent also can have hydrophilic groups that orient towards the aqueous solution.

An agent can have surface-active properties. Monomers of agent can form a monolayer at the liquid-air interface, thus reducing the surface tension of the liquid. The formation of non-equilibrium aggregates alters the surface tension of the solutions by reducing the number of monomers available to form the monolayer at the surface of the liquid. As the non-equilibrium aggregates disperse to monomers over time, the surface tension is altered. An agent also can have an indirect effect on surface tension. For example, an agent can form non-equilibrium aggregates such that the size and/or size distribution of micelles in the liquid is altered. As the agent disperses, the micelle size and/or size distribution changes, thus altering the surface tension of the composition.

The tendency of an agent to form aggregates can be known or can be determined empirically. Aggregation properties can be predicted from the molecular structure of an agent. For example, an agent can have features similar to a gemini surfactant and/or a bile acid. Additionally, features including, but not limited to, rigid planar structures, including planar rings, chiral structures, amphipathic structures, ring structures that include a nitrogen in the ring, hydroxyl groups primarily on one face of the molecule, amino side groups, double bonded oxygens contributing pi electrons that participate in intermolecular interactions and hydrophobic side chains can contribute to aggregation formation. The structures interact, e.g., through hydrophobic interactions that minimize interaction with water, by hydrogen bonding, and by Van Der Waals and London dispersion interactions.

Aggregation properties of an agent also can be determined by empirical methods. For example, an agent is mixed in a liquid composition and the aggregation state is measured by static or dynamic light scattering measurements, NMR, or any other means known in the art for measuring aggregation state of a molecule, including surface tension measurements. Aggregation state can be compared at different time points to determine whether the composition is at equilibrium or whether the aggregation state changes over time. For non-equilibrium aggregates, the aggregation state changes over time until eventually an equilibrium is achieved.

Effects on surface tension also can be determined empirically. Surface tension can be measured at different time points. For example, surface tension can be measured immediately following formulation of the composition and at a later time point. Compositions at equilibrium have a constant surface tension when compared under the same conditions (e.g. same temperature and pressure). Non-equilibrium compositions can have a surface tension that changes over time.

5. Exemplary Agents

The compositions herein have a predetermined surface tension and can include exemplary agents described herein and any other agents known in the art, for which a composition at a predetermined surface tension is desired. Exemplary agents include pharmaceutical agents that have a tendency to form aggregates, including pre-micellar aggregates, and other small and large non-equilibrium aggregates in a composition of liquid form. Exemplary agents include those that form dimers, trimers and other oligomers. Such compositions can be manufactured to have a stable predetermined surface tension using the methods of manufacture provided herein.

Compositions provided herein contain one or more agents used in the treatment of a disease or a condition. In one embodiment, the composition is effective in treating an infection or an inflammation. For example, compositions include compositions effective to treat sinusitis, including acute, recurring and chronic sinusitis. In another embodiment, the composition is effective in treating a disease or condition where such treatment includes penetration, adhesion or retention of the composition or one or more agents of a composition on a bodily surface, for example a mucosal surface, such as the nasal sinuses.

Exemplary agents include anti-infectives, including agents with antibacterial and antifungal activities, anti-inflammatories, mucolytic agents, antihistamines, antileukotrienes, decongestants, cannabinoids and anticholinergic agents. Exemplary anti-infectives include, but are not limited to, aminoglycosides, penicillins, quinolones, cephalosporins, macrolides, vancomycin, and antifungals, including but not limited to amphotericin, itraconazole, and fluconazole. Exemplary anti-inflammatories, include but not limited to, steroids, including glucocorticoids, such as betamethasone phosphate, fluticasone, and mometasone.

Compositions and methods of manufacturing compositions include compositions with one, two or more agents. Such agents can include agents from the same category (e.g. two or more aminoglyosides) and compositions containing agents from different compound classes (e.g. aminoglycosides and steroids).

a. Aminoglycosides

Among the compositions included herein are compositions of an amino glycoside formulated to have a predetermined surface tension. Aminoglycosides are molecules with an amino sugar and an amino- or guanido-substituted inositol rings which are attached by a glycosidic linkage to a hexose nucleus resulting in a polycationic and highly polar compound. Exemplary aminoglycosides include streptomycin, gentamicin, amikacin, kanamycin, tobramycin, netilmicin, neomycin, framycetin, dihydrostreptomycin, dibekacin, streptomycin and paromomycin.

Compositions can include one or more aminoglycosides that have a tendency to form non-equilibrium aggregates. Structures including planar rings, chiral structures, rings incorporating nitrogen, hydroxyl groups oriented for hydrogen bonding and amino side groups can promote aggregation. Two or more molecules of an aminoglycoside can interact orienting the more hydrophobic face of the molecule inward, away from solution and the more hydrophilic face towards the solution. An aminoglycoside also can interact with surfactants present in the solution to form pre-micelle aggregates and/or vesicles.

Pharmaceutical compositions of aminoglycosides can exhibit variation in surface tension under particular conditions of manufacture. For example, the compositions can include one or more aminoglycosides that have a tendency to form non-equilibrium aggregates that can alter surface tension. Such effects on surface tension can be further magnified in compositions that have low concentrations of other surface active molecules such as low concentrations of surfactants. Using methods herein, compositions of aminoglycosides can be manufactured such that the manufactured compositions have a stable predetermined surface tension.

In one embodiment, a composition is manufactured with tobramycin as a pharmaceutical agent in an aqueous composition. Tobramycin can form non-equilibrium aggregates in solution, for example, tobramycin dimers. The structure of tobramycin can be compared to a hybrid of a gemini surfactant and a bile salt. Tobramycin contains four amino groups that extend outwards on the same face of the molecule. Tobramycin also contains numerous hydroxyl groups. Two molecules of tobramycin can be arranged in a dimer such that the majority of the hydroxyl groups are sandwiched between the two molecules and can interact through hydrogen bond formation. The amino groups of each tobramycin molecule in the dimer face outwards towards the solution. In solutions containing nonionic surfactant, tobramycin also can interact with surfactant molecules to form pre-micelle aggregates and small, vesicular micelles.

Tobramycin can form a monolayer at the air-water interface of a solution. Dimer molecules of tobramycin act as non-equilibrium pre-micellar aggregates. Dimers of tobramycin, like other aggregates, do not participate in the monolayer formed at the air-water interface. As the number of aggregates increases, the number of tobramycin monomers that participate in monolayer formation are reduced. Aggregates containing nonionic surfactant and tobramycin also prevent monolayer formation. These aggregates have a higher surface charge and are therefore more polar than either the monomer or a larger, stable micelle, causing an increase in surface tension. Also, due to the effect at the air-water interface monolayer, surface tension of solutions with tobramycin aggregates is increased. Tobramycin aggregates, such as tobramycin dimers, and mixed tobramycin:surfactant aggregates are not equilibrium structures in solution. Over time, tobramycin partitions into stable mixed micelles or the aggregates can disperse into tobramycin monomers. Consequently, over time, surface tension of the solution can decrease.

Pharmaceutical compositions of tobramycin can exhibit variation in surface tension following manufacture, for example, as a result of the tendency of tobramycin to form pre-micellar aggregates. Such effects on surface tension can be further magnified in tobramycin compositions that have low concentrations of other surface-active molecules such as low concentrations of surfactants. Low concentrations of surfactants in such compositions can be advantageous in reducing side effects such as irritation and dryness upon administration of the composition to a subject.

b. Penicillins

Among the compositions included herein are compositions of a penicillin formulated to have a predetermined surface tension. Penicillins also include synthetic derived molecules. Exemplary penicilins include, but are not limited to, nafcillin, ticarcillin, clavulanic acid, amoxicillin, ampicillin, bacampicillin, carbenicillin cloxacillin, dicloxacillin, flucloxacillin, methicillin, meziocillin, oxacillin, penicillin G, penicillin V, piperacillin, pivampicillin, pivmecillinam and salts and derivatives thereof. Examples of salts and derivatives include, but are not limited to, aluminum penicillin, benzathine penicillin, benzyl penicillin potassium, benzyl penicillin sodium, clemizole penicillin, dimethoxyphenyl penicillin sodium, penicillin G, penicillin G benzathine, penicillin G potassium, penicillin G procaine, penicillin G sodium, isoxazolyl penicillins, including oxacillin, cloxacillin, and dicloxacillin, penicillin N, penicillin O, penicillin O potassium, penicillin O sodium, phenoxymethyl penicillin, potassium phenoxymethyl penicillin, penicillin V, penicillin V benzathine, and penicillin V potassium.

Penicillins can be classified into four broad categories, each covering a different spectrum of activity. The natural penicillins (penicillin G and penicillin V) have activity against many gram-positive organisms, gram-negative cocci, and some other gram-negative organisms. The aminopenicillins (ampicillin, amoxicillin, bacampicillin, and pivampicillin) have activity against penicillin-sensitive gram-positive bacteria, as well as Escherichia coli, Proteus mirabilis, Salmonella sp., Shigella sp., and Haemophilus influenzae. The antistaphylococcal penicillins (cloxacillin, dicloxacillin, flucloxacillin, methicillin, nafcillin, and oxacillin) also are active against beta-lactamase—producing staphylococci. The antipseudomonal penicillins (carbenicillin, meziocillin, piperacillin, and ticarcillin) have less activity against gram-positive organisms than the natural penicillins or aminopenicillins; however, unlike the other penicillins, these penicillins are active against some gram-negative bacilli, including Pseudomonas aeruginosa. Another penicillin, which does not neatly fit into any of these four categories, is pivmecillinam. Pivmecillinam is hydrolyzed during absorption to liberate the active agent, mecillinam. Mecillinam has poor activity against gram-positive organisms, Haemophilus, and Neisseria species; however, it has very good activity against many gram-negative bacteria, including E. coli, many Klebsiella, Enterobacter, and Citrobacter species. It has variable activity against Proteus sp. and does not inhibit P. aeruginosa or anaerobes, such as B. fragilis or Clostridium species.

Compositions for treatment of specific diseases and conditions can include one or more penicilins with activity against the infecting organism(s). For example, in the treatment of chronic sinusitis, penicillins including amoxicillin, ampicillin, bacampicillin, cloxacillin, flucloxacillin, methicillin, nafcillin, oxacillin, ticaracillin and penicillin V can be formulated in compositions for manufacture and administration as described herein.

The compositions can include one or more penicillins that have a tendency to form non-equilibrium aggregates. Penicillins can form small non-equilibrium aggregates in liquid compositions, including dimers, trimers and other small aggregates. Structures including long-side chains and ring structures containing nitrogens can contribute to the aggregation properties of penicillins. Pharmaceutical compositions of penicillins can exhibit variation in surface tension following manufacture, for example, as a result of the ability of the molecules to form non-equilibrium aggregates. Such effects on surface tension can be further magnified in compositions that have low concentrations of other surface-active molecules such as low concentrations of surfactants. Using methods herein, compositions of penicillins can be manufactured such that the manufactured compositions have a stable predetermined surface tension. The manufactured compositions maintain a predetermined surface tension from manufacture through storage and administration.

c. Quinolones

Among the compositions included herein are compositions of a quinolone formulated to have predetermined surface tension. Quinolones are antibiotic agents. Exemplary quinolones include, but are not limited to, ofloxacin, levofloxacin, ciprofloxacin, norfloxacin, enoxacin, lomefloxacin, grepafloxacin, trovafloxacin, sparfloxacin, temafloxacin, moxifloxacin, and gatifloxacin. Newer classes of quinolones including fluoroquinolones have a broad spectrum of activity. For example, second-generation quinolones have increased gram-negative activity, as well as some gram-positive and atypical pathogen coverage as compared with first-generation drugs. Second-generation agents include ciprofloxacin, enoxacin, lomefloxacin, norfloxacin and ofloxacin. Ciprofloxacin is the most potent fluoroquinolone against P. aeruginosa. Third-generation quinolones, including levofloxacin, gatifloxacin, moxifloxacin and sparfloxacin, have expanded activity against gram-positive organisms, particularly penicillin-sensitive and penicillin-resistant S. pneumoniae, and atypical pathogens such as Mycoplasma pneumoniae and Chlamydia pneumoniae.

Compositions for treatment of specific diseases and conditions can include one or more quinolones with activity against the infecting organism(s). For example, in the treatment of sinusitis, ciprofloxacin, levofloxacin, gatifloxacin, moxifloxacin, trovafloxacin are used.

The compositions can include one or more quinolones that have a tendency to form non-equilibrium aggregates. Quinolones can form non-equilibrium aggregates in liquid compositions. Structures including a rigid planar double-ring structure, rings that contain nitrogen and double bonded oxygens contributing pi electrons for intermolecular interactions can contribute to the aggregation properties of quinolones. Pharmaceutical compositions of quinolones can exhibit variation in surface tension following manufacture, for example, as a result of the tendency of the molecules to form non-equilibrium aggregates. Such effects on surface tension can be further magnified in compositions that have low concentrations of other surface-active molecules such as low concentrations of surfactants. Using methods herein, compositions of quinolones can be manufactured such that the compositions have a stable predetermined surface tension. The manufactured compositions maintain a predetermined surface tension from manufacture through storage and administration.

d. Cephalosporins

Among the compositions included herein are compositions of a ephalosporin formulated to have a predetermined surface tension. Exemplary cephalosporins include, but are not limited to, cefuroxime, ceftazidime, ceftriaxone, cefotaxime, cefoxitin, cefazolin, cephalexin, cephradine, cefadroxil, loracarbef, cefprozil, cefaclor, cefotetan, cefamandole, cefixime, cefpodoxime, and cefizoxime. Compositions for treatment of specific diseases and conditions can include one or more cephalosporins with activity against the infecting organism(s). For example, in the treatment of sinusitis, cefuroxime, ceftazidime, ceftriaxone, cefotaxime, cefoxitin, cefazolin are used.

The compositions can include one or more cephalosporins that have a tendency to form non-equilibrium aggregates, including pre-micellar aggregates. Structures including a rigid planar structure and hydrophobic side chains for intermolecular interactions can contribute to the aggregation properties of cephalosporins. Pharmaceutical compositions of cephalosporins can exhibit variation in surface tension following manufacture, for example, as a result of the tendency of the molecules to form non-equilibrium aggregates. Such effects on surface tension can be further magnified in compositions that have low concentrations of other surface-active molecules such as low concentrations of surfactants. Using methods herein, compositions of cephalosporins can be manufactured such that the compositions have a stable predetermined surface tension. The manufactured compositions maintain a predetermined surface tension from manufacture through storage and administration.

e. Anti-Fungal Agents

Among the compositions herein are compositions of anti-fungal agents formulated to have a predetermined surface tension. Exemplary anti-fungal agents include, but are not limited to non-azole agents including amphotericin B, polyenes such as nystatin, allylamines such as naftifine and terbinafine, pyrimidine analogs, such as flucytosine (5-flurocytosine), griseofulvin, haloprogin, and tolnaftate. Exemplary azole antifungal agents include, but are not limited to, butoconazole, clotrimazole, econazole, fluconazole, itraconazole, ketoconazole, miconazole, oxiconazole, sulconazole and terconazole.

Compositions for treatment of specific diseases and conditions can include one or more anti-fungal agents with activity against the infecting organism(s). For example, in the treatment of sinusitis, amphotericin and itraconazole are most commonly used.

The compositions can include one or more anti-fungal agents that have a tendency to form non-equilibrium aggregates. Structures including multi-ring structures, large-ring structures, structures resembling nucleosides and double-bonded oxygens can contribute to the aggregation properties of anti-fungal agents. Pharmaceutical compositions of anti-fungal agents can exhibit variation in surface tension following manufacture, for example, as a result of the tendency of the molecules to form non-equilibrium aggregates. Such effects on surface tension can be further magnified in compositions that have low concentrations of other surface-active molecules such as low concentrations of surfactants. Using methods herein, compositions of anti-fungal agents can be manufactured such that the compositions have a stable predetermined surface tension. The manufactured compositions maintain a predetermined surface tension from manufacture through storage and administration.

f. Additional Classes of Anti-Infective Agents

The compositions herein can include additional classes of anti-infective agents formulated to have a predetermined surface tension. The compositions can include anti-infective agents that have a tendency to form aggregates, including pre-micellar aggregates. For example, vancomycin, a large, multi-ring structure, can form aggregates. Additionally, compositions can include anti-infectives in the macrolide class of molecules. Exemplary macrolides include clindamycin and azithromycin. Pharmaceutical compositions of anti-infective agents such as vancomycin and macrolides can exhibit variation in surface tension following manufacture, for example, as a result of the tendency of the molecules to form aggregates. Such effects on surface tension can be further magnified in compositions that have low concentrations of other surface-active molecules such as low concentrations of surfactants. Using methods herein, compositions of anti-infective agents such as vancomycin and macrolides can be manufactured such that the compositions maintain a predetermined surface tension from manufacture through storage and administration.

g. Anti-Inflammatory Steroidal Agents

Among the compositions herein are compositions of anti-inflammatory agents including steroids, formulated to have a predetermined surface tension. Exemplary anti-inflammatory agents include, but are not limited betamethasone, fluticasone, mometasone, triamcinolone, dexamethasone, prednisone, beclomethasone, flunisolide, and budesonide. Compositions for treatment of specific diseases and conditions can include one or more anti-inflammatories with activity for treatment of inflammation associated with the disease or condition. For example, in the treatment of sinusitis, steroids including betamethasone, fluticasone and mometasone are used.

The compositions can include one or more anti-inflammatory agents that can form non-equilibrium aggregates. Structures including a rigid planar multi-ring structure and structural similarities with bile acid salts, the cholesterol-derived sterol nucleus can contribute to the aggregation properties of anti-inflammatories, such as steroids. Pharmaceutical compositions of anti-inflammatories can exhibit variation in surface tension following manufacture. For example, as a result of the tendency of the molecules to form non-equilibrium aggregates under particular conditions, the surface tension of the composition can be altered. Such effects on surface tension can be further magnified in compositions that have low concentrations of other surface-active molecules such as low concentrations of surfactants. Using methods herein, compositions of anti-inflammatories can be manufactured such that the compositions have a stable predetermined surface tension. The manufactured compositions maintain a predetermined surface tension from manufacture through storage and administration.

h. Anti-Inflammatory Non-Steroidal Agents

Among the compositions herein are compositions of anti-inflammatory agents including non-steroidal anti-inflammatory drugs (NSAIDS) formulated to have a predetermined surface tension. Exemplary NSAIDs include, but are not limited to diclofenac, acetylsalicylic acid, methyl salicylate, ibuprofen, ketoprofen, naproxen, nabumetone, oxaprozin, rofecoxib, phenacetin, butacetin, acetaminophen, acetamidoquinone, nefopam, fenoprofen, flurbiprofen, etodolac, indomethacin, ketorolac, sulindac tolmetin meclofenamate, mefenamic acid, piroxicam and suprofen. Compositions for treatment of specific diseases and conditions can include one or more NSAIDs with anti-inflammatory activity for treatment of inflammation associated with the disease or condition. For example, in the treatment of sinusitis, NSAIDs including diclofenac are used.

The compositions can include one or more NSAID agents that have a tendency to form non-equilibrium aggregates. Structures including a rigid planar ring or multi-ring structures and hydroxyl groups for hydrogen bonding, and double-bonded oxygens can contribute to the aggregation properties of NSAIDs. Pharmaceutical compositions of NSAIDs can exhibit variation in surface tension following manufacture. For example, as a result of the tendency of the molecules to form non-equilibrium aggregates, the surface tension of the composition can be altered. Such effects on surface tension can be further magnified in compositions that have low concentrations of other surface-active molecules such as low concentrations of surfactants. Using methods herein, compositions of NSAIDs can be manufactured such that the compositions have a stable predetermined surface tension. The manufactured compositions maintain a predetermined surface tension from manufacture through storage and administration.

i. Antihistamines

Among the compositions herein are compositions of antihistamine agents formulated to have a predetermined surface tension. Compositions for treatment can include one or more antihistamine agents. Antihistamines help relieve nasal allergy symptoms such as: congestion, itching, and discharge; eye symptoms such as itching, burning, tearing, clear discharge; skin conditions such as hives, eczema, itching and some rashes; and other allergic conditions. Antihistamines can relieve symptoms of allergy accompanying a cold, or they can have an anticholinergic effect that dries cold secretions. Exemplary classes of antihistamines include, but are not limited to, ethanolamines, ethylenediamines, alkylamines, piperzines, and phenothiazines. Exemplary antihistamine agents include, but are not limited to fenethazine, loratadine, cyproheptadine, diphenhydramine, dimenhydrinate, doxylamine, chlorpheniramine, brompheniramine, cinnanizine, fexofenadine, cetirizine, terfenadine, hydroxyzine, azelastine, and cromolyn.

The compositions can include one or more antihistamine agents that have a tendency to form non-equilibrium aggregates. Structures including planar rings, nitrogens in the ring structures and double bonded oxygens can contribute to the aggregation properties of antihistamine agents. Pharmaceutical compositions of antihistamine agents can exhibit variation in surface tension following manufacture, for example, as a result of the tendency of the molecules to form non-equilibrium aggregates. Such effects on surface tension can be further magnified in compositions that have low concentrations of other surface-active molecules such as low concentrations of surfactants. Using methods herein, compositions of antihistamine agents can be manufactured such that the compositions have a stable predetermined surface tension. The manufactured compositions maintain a predetermined surface tension from manufacture through storage and administration.

j. Antileukotrienes

Among the compositions herein are compositions of antileukotriene agents formulated to have a predetermined surface tension. Leukotrienes play a key role in inflammatory responses and are involved in generating many different inflammatory pathologies. Leukotrienes are produced and released from inflammatory cells, including eosinophils and mast cells. The release of leukotrienes from inflammatory cells induces bronchoconstriction, mucous secretion, and increased vascular permeability. Antileukotrienes that block leukotrienes at the receptor level have been shown to be relatively safe and effective in the treatment of chronic mild to moderate asthma. Compositions for treatment of specific diseases and conditions can include one or more antileukotriene agents with anti-inflammatory activity. For example, compositions containing zileuton (5-lipoxygenase inhibitor), zafirlukast, montelukast, and pranlukast can be used in the treatment of asthma and rhinosinusitis

The compositions can include one or more antileukotriene agents that have a tendency to form non-equilibrium aggregates. Structures including planar rings, nitrogens in the ring structures and double bonded oxygens can contribute to the aggregation properties of antileukotrienes. Pharmaceutical compositions of antileukotriene agents can exhibit variation in surface tension following manufacture. For example, as a result of the tendency of the molecules to form non-equilibrium aggregates, the surface tension of a composition can be altered. Such effects on surface tension can be further magnified in compositions that have low concentrations of other surface-active molecules such as surfactants. Using methods herein, compositions of antileukotriene agents can be manufactured such that the compositions have a stable predetermined surface tension. The manufactured compositions maintain a predetermined surface tension from manufacture through storage and administration.

k. Decongestants

Among the compositions herein are compositions of decongestants formulated to have a predetermined surface tension. Exemplary decongestants agents include, but are not limited to naphazoline, albuterol, isoetharine, tetrabutaline, metaproterenol, phenylpropanolamine, pseudoephedrine, phenylephrine, epinephrine, ephedrine, desoxyephedrine, naphazoline, oxymetazoline, tetrahydrozoline, xylometazoline and propylhexedrine. Compositions for treatment of specific diseases and conditions can include one or more decongestants with activity against symptoms of such diseases and conditions, including nasal and respiratory congestion. For example, decongestants can be administered in acute conditions such as hay fever, allergic rhinitis, vasomotor rhinitis, sinusitis and the common cold to relieve membrane congestion. For example, in the treatment of sinusitis phenylephrine, naphazoline, oxymetazoline, tetrahydrozoline, and xylometoazoline are typically used.

The compositions can include one or more decongestants that have a tendency to form non-equilibrium aggregates. Structures including planar rings, multi-ring structures, nitrogens in the ring structures and double bonded oxygens can contribute to the aggregation properties of decongestants. Pharmaceutical compositions of decongestants can exhibit variation in surface tension following manufacture, for example, as a result of the tendency of the molecules to form non-equilibrium aggregates. Such effects on surface tension can be further magnified in compositions that have low concentrations of other surface-active molecules such as low concentrations of surfactants. Using methods herein, compositions of decongestants can be manufactured such that the compositions have a stable predetermined surface tension. The manufactured compositions maintain a predetermined surface tension from manufacture through storage and administration.

l. Antivirals

Among the compositions herein are compositions of antiviral agents formulated to have a predetermined surface tension. Exemplary antiviral agents include, but are not limited to amantadine analogs such as amantadine, rimantidine, adamantine, and memantine, hydroxybenzimidazole, nucleoside analogs such as cytarabine, vidarabine, acyclovir, ganciclovir, famciclovir, penciclovir, valacyclovir, and zanavimir, dideoxynucleosides including azidothymidine, dideoxyinosine, dideoxyadenine and dideoxycytidine, and late-stage inhibitors such as flurophenylalaine, puromycin, methiszaone, and rifampin.

The compositions can include one or more antiviral agents that have a tendency to form non-equilibrium aggregates. Structures including planar rings, nitrogens in the ring structures and double bonded oxygens as well as structural similarities to nucleosides that have stacking properties can contribute to the aggregation properties of antiviral agents. Pharmaceutical compositions of antiviral agents can exhibit variation in surface tension following manufacture, for example, as a result of the tendency of the molecules to form non-equilibrium aggregates. Such effects on surface tension can be further magnified in compositions that have low concentrations of other surface-active molecules such as low concentrations of surfactants. Using methods herein, compositions of antiviral agents can be manufactured such that the compositions have a stable predetermined surface tension. The manufactured compositions maintain a predetermined surface tension from manufacture through storage and administration.

m. Anesthetics

Among the compositions herein are compositions of anesthetic agents formulated to have a predetermined surface tension. Exemplary anesthetic agents include, but are not limited to procaine analogs, such as procaine (Novocaine), bezocaine, siocaine, oxycaine and lidocaine. Compositions for treatment can include one or more anesthetic agents with activity as a local anesthetic.

The compositions can include one or more anesthetic agents that have a tendency to form non-equilibrium aggregates. Structures including planar rings, polar groups and double bonded oxygens can contribute to the aggregation properties of anesthetic agents. Pharmaceutical compositions of anesthetic agents can exhibit variation in surface tension following manufacture, for example, as a result of the tendency of the molecules to form non-equilibrium aggregates. Such effects on surface tension can be further magnified in compositions that have low concentrations of other surface-active molecules such as low concentrations of surfactants. Using methods herein, compositions of anesthetic agents can be manufactured such that the compositions have a stable predetermined surface tension. The manufactured compositions maintain a predetermined surface tension from manufacture through storage and administration.

6. Additional Ingredients of the Compositions

Compositions provided herein include one or more agents formulated in a liquid form. Liquid forms include solutions, suspensions and emulsions. The compositions can be aqueous or non-aqueous compositions. Generally, the compositions are aqueous. The compositions also can include one or more additional ingredients, including, but not limited to, surfactants, one or more additional agents buffers, salts, flavorings and preservatives.

a. Surfactants

Compositions can contain one or more surfactants. Surfactants can be used as dispersing agents, solubilizing agents, and spreading agents. Surfactants also can be used to lower the surface tension of a composition. At levels below the critical micelle concentration, addition of such surfactants lowers surface tension. Above the critical micelle concentration, addition of surfactant does not measurably alter the surface tension of the composition.

In an example of the compositions provided herein, surfactants are added near the critical micelle concentration (cmc), such that the amount of free surfactant (e.g. surfactant not found in micelles) is maximized. Near the cmc includes concentrations equal to the cmc and concentrations within 2 times and 3 times the cmc, including concentrations above and below the cmc. For example, if the cmc for a surfactant is 0.06 mM, near the cmc can include 0.01 mM, 0.02 mM, 0.03 mM, 0.04 mM, 0.05 mM, 0.06 mM, 0.07 mM, 0.08 mM, 0.09 mM, 0.10 mM, 0.11 mM, 0.12 mM, 0.13 mM, 0.14 mM, 0.15 mM, 0.16 mM, 0.17 mM, 0.18 mM and any concentrations in between. Formulation of a composition with a concentration of surfactant near the cmc maximizes the contribution of the surfactant to surface tension activity of the composition.

The amount of surfactant can be adjusted such that the composition has a predetermined surface tension. In one example, the predetermined surface tension of the compositions is between 10 dynes/cm and about 70 dynes/cm. In another example, the predetermined surface tension of the compositions is between 20 dynes/cm and about 60 dynes/cm. In another example, the predetermined surface tension of the compositions is between 30 dynes/cm and about 50 dynes/cm. In another example, the predetermined surface tension of the compositions is between 35 dynes/cm and about 45 dynes/cm.

In one embodiment, the predetermined surface tension is chosen to effect deposition, penetration and/or retention of the composition or an agent contained in the composition onto a bodily surface upon administration of the composition to a subject. For example, the predetermined surface tension effects deposition, penetration and/or retention of the composition or an agent contained in the composition in the nasal sinuses.

Surfactants can be used over a broad range of concentrations. For solubilization of other molecules, such as agents, surfactant concentrations generally fall between 1% and 20% by weight of the composition. Concentration of surfactant can be tailored to reduce unwanted side effects that often occur from treatments with compositions containing higher levels of surfactant. Such side effects include drying of the mucosa, epistaxis, and irritation. Compositions that contain low concentrations of surfactant can be used to reduce side effects. Low concentrations of surfactant refer to surfactant concentrations that are near the cmc. Such concentrations are usually below the concentrations typically used for solubilization of an agent. Generally, low surfactant concentrations are below 1% by weight. For instance, low concentrations of surfactant include concentrations of 0.9%, 0.8%, 0.6%, 0.5%, 0.4%, 0.3%, 0.2%, 0.1%, 0.08%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001% and less than 0.001% by weight. In one example, a composition contains surfactant at a concentration between 0.009% and 0.015% by weight. The amount of surfactant will depend on the surfactant, the ingredients of the composition, the mode of administration and the desired predetermined surface tension. The concentration of surfactant can be determined empirically. In one example, compositions contain a low concentration of surfactant that includes a concentration near the cmc.

Surfactants that can be used herein include anionic, cationic, zwitterionic, gemini and nonionic surfactants. These include, but are not limited to, polyoxyethylene-sorbitan-fatty acid esters, hydrogenated castor oils, castor oil derivatives such as polyethyleneglycol (PEG)-castor oils, sorbitan fatty acid esters, polyalkylene glycols, polyoxyethylene nonyl phenols, polyoxyethylene alkyl ethers, octylphenol ethoxylates, tyloxapol (also known as oxyethylated tertiary octylphenol-polymethylene polymer), and sodium lauryl sulfate. These surfactants are further described in “Handbook of Pharmaceutical Excipients”, 2nd Edition, Editors A. Wade and P. J. Weller (1994), published by American Pharmaceutical Association, Washington, USA and The Pharmaceutical Press, London, England. Exemplary surfactants include, but are not limited to PEG 400, sodium lauryl sulfate, sorbitan esters (Span®-20, Span®-40, Span®-60, etc.), polysorbates (Tween®-20, Tween®-40, Tween®-60, etc.), tyloxapol, and propylene glycol. In one embodiment, the surfactant in the composition is a non-ionic surfactant. Non-ionic surfactants include octylphenol ethoxylates (e.g. Triton®X-100), polysorbates, polyethylene glycols (PEGs), hydrogenated castor oils and polyethyleneglycol castor oils (Cremophor®s) and polyoxyethylene alkyl ethers (Brij®-30, Brij®-35, Brij®-52, Brij®-56, etc.). In one embodiment, the compositions contain polysorbate-20. For the purposes and compositions herein, benzalkonium chloride is not considered a surfactant.

Polyoxyethylene-sorbitan-fatty acid esters include, for example, mono- and tri-lauryl, palmityl, stearyl and oleyl esters of the type known and commercially available, such as under the trade name Tagat® and Tween®. These include Tween®-20 (polyoxyethylene(20)sorbitan monolaurate), Tween®-21 (polyoxyethylene(4)sorbitan monolaurate), Tween®-40 (polyoxyethylene(20)sorbitan monopalmitate), Tween®-60 (polyoxyethylene(20)sorbitan monostearate), Tween®-65 (polyoxyethylene(20)sorbitan tristearate), Tween®-80 (polyoxyethylene(20)sorbitan monooleate), Tween®-81 (polyoxyethylene(5)sorbitan monooleate], and Tween®-85 (polyoxyethylene(20)sorbitan trioleate).

Castor oil derivatives include hydrogenated castor oil and the reaction products of a natural or hydrogenated castor oil with ethylene oxide. The hydrogenated castor oils are commercially available, such as under the trade name Cremophor®, such as Cremophor®RH 40 and Cremophor®RH 60. Polyethyleneglycol castor oils are commercially available, such as under the trade name Cremophor®, such as Cremophor® EL and under the trade name Incrocas®, such as Incrocas®-35. Other polyoxyethyleneglycol-alkylethers are available commercially, such as under the trade name Nikkol®.

Sorbitan fatty acid esters include sorbitan mono C₁₂₋₁₈ fatty acid esters, or sorbitan tri C₁₂₋₁₈ fatty acid esters as known and commercially available, such as sorbitan laurate, sorbitan palmitate, sorbitan stearate, including those sold under the trademark Span®, such as Span®20 (sorbitan monolaurate), Span®40 (sorbitan monolaurate), Span®60 (sorbitan monostearate), Span®65 (sorbitan tristearate) or Span®80 (sorbitan monooleate). Examples of polyalkylene glycol materials are polyethylene glycols (PEGs), in particular polyethylene glycols having a molecular weight of from about 400 to about 4,000. Polyoxyethylene nonyl phenols are available commercially, such as under the trade name Igepal®. Polyoxyethylene alkyl ethers, such as polyoxyethylene stearyl ethers, are available commercially under the trademark Brij®. Octylphenol ethoxylates are commercially available, such as under the trademark Triton® (for example, Triton® X-100). Tyloxapol is available commercially, such as Triton® WR-1339.

In another embodiment, more than one surfactant is contained in the composition. In one such example, two or more surfactants are added to obtain a predetermined hydrophile-lipophile balance (HLB). The HLB is used to describe the characteristics of a surfactant. The system includes an arbitrary scale to which HLB values are experimentally determined and assigned. If the HLB value is low, the number of hydrophilic groups on the surfactant is small and it is more lipophilic (oil soluble). An HLB value of 10 or higher means that the agent is primarily hydrophilic, while an HLB value of less than 10 means it would be lipophilic. For example, sorbitan fatty acid esters, such as Span®s, have HLB values ranging from 1.8 to 8.6, which is indicative of oil soluble for oil dispersible molecules. Polyoxyetheylene sorbitan fatty acid esters, such as the polysorbates sold as Tween®s, have HLB values that range from 9.6 to 16.7, which is characteristic of water-soluble or water dispersible molecules.

Low concentrations of surfactants can be advantageous in reducing side effects such as irritation and dryness upon administration of the composition to a subject. Low concentrations include concentrations of surfactant near or at the critical micelle concentration of the surfactant. In an example of the compositions provided herein, the compositions contain a surfactant at a concentration at or near the cmc of the surfactant and one or more agents that have a tendency to form non-equilibrium aggregates. The surfactant concentration is adjusted to be at or near the cmc such that the composition has a predetermined surface tension. Additionally, the low amount of surfactant reduces side effects of the composition when administered. The methods of manufacture herein reduce the formation of non-equilibrium aggregates of an agent such that such aggregates do not alter the surface tension. The methods produce equilibrium compositions of an agent, for example, an agent and a surfactant in a liquid, such that the composition has a predetermined surface tension that remains stable over time.

b. Salt

Salt concentrations can be adjusted to provide a predetermined osmolality of the compositions. In addition, salt concentrations and types of salts can be tailored for particular uses of compositions. Salt concentrations can include the concentration of added salts such as NaCl, and agents or other ingredients that contribute to the overall salt concentration of the composition. This overall salt concentration can be expressed as NaCl equivalency.

Optimal NaCl equivalency for a particular composition can be known or determined empirically. For particular embodiments, a desired NaCl equivalency can be selected or pre-selected. For example, in compositions for application to the nasal mucosa, such as in compositions for treatment of chronic sinusitis, NaCl equivalency of the composition is hypotonic or isotonic. This NaCl equivalency can reduce side-effects upon administration, including reducing swelling in the sinuses and nasal cavity and avoiding burning sensation of the administered composition.

Osmotic pressure (osmolality) is related to the concentration of solutes in solution and is influenced by the temperature of the solution. Osmotic pressure can be measured by using an Osmometer. If necessary, osmotic pressure can be adjusted to fall within a preferred range by adding NaCl, dextrose, or other salts to the composition. Osmotic pressure of a composition can be tailored for the active agent, disease or condition to be treated and route of administration. For example, for hypo-osmotic and iso-osmotic compositions used to treat chronic sinusitis, typically such compositions have an osmolality between 150 to 450 Osm/kg. In one example, the osmolality is between 160 to 240 Osm/kg. Hyperosmotic compositions can have a higher osmolality.

Salt concentration affects the cmc of a surfactant in solution. For example, an increase in salt concentration decreases the cmc of a non-ionic surfactant. Salt concentrations can be determined empirically to achieve a predetermined surface tension by formulating the desired agents and ingredients, including salt and determining the surface tension. Concentrations of one or more ingredients can be adjusted or addition of other ingredients can be used to adjust the surface tension of the composition to achieve a predetermined surface tension.

c. pH

Compositions can be adjusted to be in a desired pH range. The effectiveness of an agent and/or the stability of a particular composition can be affected by pH (see for example American Hospital Formulary Service (AFHS) published yearly and the Hand Book of Injectable Drugs by Lawrence A. Trissel, 1994 American Society of Hospital Pharmacists, Inc., incorporated by reference herein, regarding stability or effectiveness of a medication at certain pH). In addition, pH can affect the solubility of an agent in solution, affecting the balance of monomers and aggregates in solution. For example, as the pH becomes more acidic, agents can increase in solubility and the amount of aggregation is decreased.

The pH can be measured with any available assay known in the art including pH meters and strips. The pH of a composition can be adjusted with buffering agents, including acids and bases to reach the desired pH range.

d. Solubility and Dispersal

Compositions can be formulated to increase the solubility or dispersal of an agent. Surfactants can be used to promote solubility and dispersal of an agent in solution. In some cases, however, it may be desired to increase solubility or dispersal without increasing the concentration of surfactant, for example, to achieve solubility and dispersal but reduce unwanted side effects such as dryness and irritation that occur with higher concentrations of surfactant. Other ingredients of a composition can be adjusted to increase solubility and dispersal. For example, the salt concentration and the pH can be adjusted to increase solubility and dispersal. Additional ingredients, such as glucose and mannose, can be added to increase solubility and dispersal. Complexing agents also can be used; these agents noncovalently interact with solutes such that the resulting complex is rendered more water soluble than the solute, when not complexed. The concentration of an agent can be adjusted to increase solubility and dispersal. Formulations that increase solubility and dispersal can be determined empirically.

C. Exemplary Compositions for Treating Chronic Sinusitis

Any of the compositions herein with an agent effective to treat sinusitis can be used to provide compositions with a stable predetermined surface tension for the treatment of chronic sinusitis. Any of the manufacturing methods provided herein can be used to formulate compositions with a predetermined surface tension for the treatment of sinusitis.

Sinusitis is an inflammation of the membrane lining one or more paranasal sinuses. The sinuses are air-filled cavities in the skull (Stedman's Medical Dictionary, 27th Edition, page 1644, (1999), Lippincoft Williams & Wilkins, Baltimore, Md.). Four pairs of sinuses known as the paranasal sinuses, connect the space (known as the nasal passage) running from the nostrils and up through the nose. These four pairs of paranasal sinuses are the frontal sinuses, the maxillary sinuses, the ethmoid sinuses, and the sphenoid sinuses. They are located, respectively, in the forehead, behind the cheekbones, between the eyes, and behind the eyes. A membrane lining the sinuses secretes mucus, which drains into the nasal passage from a small channel in each sinus. Healthy sinuses are sterile and contain no bacteria. In contrast, the nasal passage normally contains many bacteria that enter through the nostrils as a person breathes.

When one or more of the sinuses becomes infected, sinusitis develops. There are three different types of sinusitis: acute, recurrent acute, and chronic. As an example, acute sinusitis is characterized as lasting less than three weeks or occurring less than four times a year and can be treated using antibiotics, leaving no damage to the linings of the sinus tissue. Recurrent acute sinusitis occurs more often but leaves no significant damage. Chronic sinusitis lasts longer than three weeks and often continues for months. In cases of chronic sinusitis, there is usually tissue damage. According to the Center for Disease Control (CDC), thirty seven million cases of chronic sinusitis are reported annually.

For effective treatment of sinusitis, particularly chronic sinusitis, compositions are designed to be deposited and/or retained in the nasal sinuses. Additionally, side-effects such as dryness and irritation should be minimized to avoid unnecessary discomfort when such compositions are administered and to encourage compliance of the subject in continuing the administrations through a full-course of treatment.

Effective treatment of sinusitis includes topical delivery of a pharmaceutical agent to the nasal cavity and sinuses by aerosolizing liquid compositions of an agent. Aerosolized particles of an agent are effective therapeutically within a preferred particle size. For example, for directing compositions to the nasal sinuses, particles with a mass median aerodynamic diameter (MMAD) of about 1.0 to 6.0 microns, for example, 1.0 to 5.0 microns, and 2.0 to 6.0 microns, are effective for deposition in the sinuses. Additionally, formulating liquid compositions with a surface tension effective for deposition, retention and penetration in the nasal sinuses results in a therapeutically effective treatment for sinusitis, including chronic sinusitis. For example, addition of a surfactant to formulations can be used to obtain a predetermined surface tension for deposition, retention, and penetration of an agent into the sinuses.

The compositions herein have a predetermined surface tension for deposition, retention and penetration of an agent in the nasal sinuses. In addition, the compositions also can include a low concentration of surfactant to achieve a predetermined surface tension and/or to contribute to other properties of the composition such as solubility. The provided compositions include compositions that have a concentration of surfactant that is near the critical micelle concentration. The concentration of surfactant in such compositions is tailored to reduce unwanted side effects including dryness and irritation that can result from application of higher concentrations of surfactant. The predetermined surface tension of the compositions is generally between about 30 dynes/cm and about 50 dynes/cm. In other examples, compositions can include those with surface tensions of about 35 to 45 dynes/cm. The predetermined surface tension is achieved by titrating surfactant to achieve the desired surface tension while using low concentrations of surfactant to reduce unwanted side effects. Such compositions include a concentration of surfactant near the cmc, generally within 2 or 3 times the cmc concentration. Such concentrations of surfactant are generally less than concentrations used for solubilization, typically less than 1%, usually less than 0.1% by weight. Surface tension of the composition can be adjusted with low concentrations of surfactants, including non-ionic surfactants such as polysorbates, polyoxyethylene alkyl ethers (Brij®), polyethylene glycols (PEGs), and hydrogenated castor oils and polyethyleneglycol castor oils (Cremophor® surfactants).

The osmolality of the compositions is hypo-osmotic between about 150 mOsm/kg to 450 mOsm/kg, and includes osmolality of about 150 mOsm/kg to 350 mOsm/kg, about 150 mOsm/kg to 250 mOsm/kg and about 200 mOsm/kg to 250 mOsm/kg. NaCl equivalency of the composition is adjusted to achieve a hypotonic or isotonic solution. Generally, NaCl equivalency is 0.5% or less than 0.5%. The pH of the composition can range preferably between about 3.0 and 8.5, but may vary according to the properties of the active agent used.

One or more agents for the treatment of chronic sinusitis include anti-infectives, including antibiotics and anti-fungal agents, anti-inflammatories and mucolytic agents, as well as antihistamines, antileukotrienes, decongestants, anticholinergics and antiseptics described herein and known in the art. Exemplary anti-infectives include aminoglycosides such as gentamicin, amikacin, and tobramycin, penicillins, such as nafcillin, ticarcillin, and piperacillin, quinolones such as levofloxacin, ciprofloxacin, gatifloxacin; cephalosporins such as ceftazidime, cefuroxime; vancomycin, amphotericin, and anti-fungal azoles, including itraconazole and fluconazole. Exemplary anti-inflammatories include betamethasone, fluticasone and mometasone.

The methods of manufacture herein produce compositions for the treatment of chronic sinusitis with a predetermined surface tension, typically between about 30 and 50 dynes/cm. The formation of non-equilibrium aggregates and/or other structures is reduced or eliminated, such that an equilibrium composition is obtained and the surface tension remains stable.

The compositions can be aseptically packaged as articles of manufacture, into single dose or multi-dose containers. Also optionally included are directions for use, including instructions for nebulization. The compositions can be provided as kits for example including directions for use and/or equipment for nebulization. Directions for use can include directions for nebulization, including particle sizes (a mass median aerodynamic diameter (MMAD)) in the size range of about 0.5 μm to about 5.0 μm in diameter for administration, for example, 0.5 to 5 μm, 1 to 5 μm, 2 to 4 μm, and 2 to 3.5 μm.

Such kits and articles of manufacture can include instructions for dilution and/or diluents. Acceptable diluents, for example, can be water, saline solution, or a mixture of water and alcohol.

D. Formulation of Compositions in Small Scale

Small-scale formulation of compositions can be used to empirically determine properties of compositions, as well as to compare properties of different formulations. Compounding pharmacies use small scale formulation to provide single dosages or a small number of dosages for one or more patients. Small-scale formulation generally includes formulation of volumes less than a liter and/or formulation by manual, non-automated processes. Small-scale formulation can be used to measure properties including surface tension, pH, osmolality, NaCl equivalency, stability, efficacy, and dosage for compositions. For example, such properties can be measured to compare different ingredients or different concentrations of the same ingredients for formulations. In one example, small scale formulation is used to determine ingredients and concentrations of ingredients to achieve a predetermined surface tension.

Small scale formulation usually involves smaller volumes of compositions, typically less then one liter, often 100 ml or less and can include single dosage formulations. Because the volumes are small, formulation steps including mixing, transferring, filtering and filling are usually done manually. If mechanical means are used, the force and pressure placed upon the composition are lower than that used in large scale manufacturing processes. Additionally, small scale formulation typically includes filling and packaging manually into containers that are filled in an open system (exposed to air) and are closed manually using a screw cap or interlocking cap container.

E. Manufacturing Processes

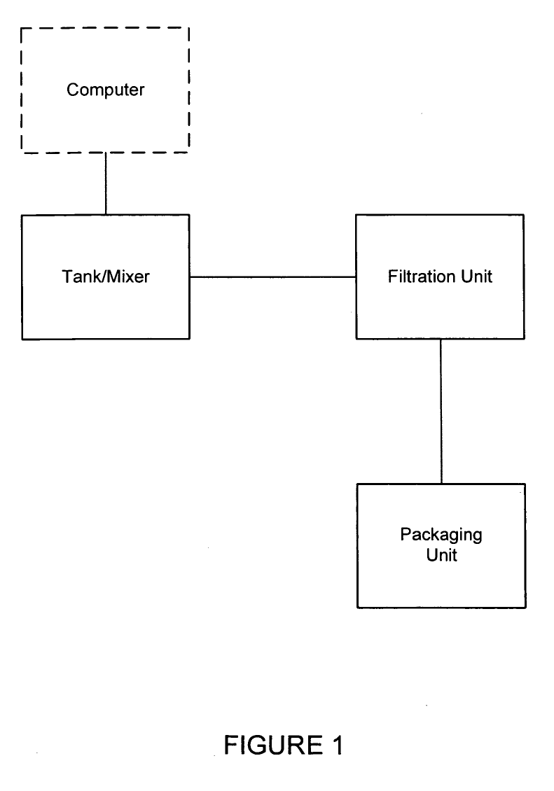

Provided herein are methods of manufacturing pharmaceutical compositions that maintain a predetermined surface tension. The general steps in a manufacturing process include, but are not limited to, mixing, transferring, and filtration of the compositions, as exemplified in FIG. 1. In one embodiment, an active agent in a liquid, such as Water for Injection (WFI), is mixed in a formulation tank, to which other composition components, such as a surfactant, is added and the composition is mixed. The pH can be adjusted during the mixing stage as required for proper formulation. The composition is pumped, for example, to a filtration unit, where the composition is then filtered, such as through a 0.22, 0.2 or 0.1 micron sterilizing filter. The sterile composition is then transported to a packaging unit, where the composition is aseptically packaged. These manufacturing process steps can be automated.

For example, in one embodiment, a computer is included to provide automated process control of the manufacturing system. Any suitable computer system may be used, and the software the controls the automated process control system may be performed on multiple computers all having a similar construction, or may be performed by a single, integrated computer. Each computer operates under control of a central processor unit (CPU), such as a “Pentium” microprocessor and associated integrated circuit chips, available from Intel Corporation of Santa Clara, Calif., USA. A computer user can input commands and data from a keyboard and display mouse and can view inputs and computer output at a display. The display is typically a video monitor or flat panel display device. The computer also includes a direct access storage device (DASD), such as a fixed hard disk drive. The memory typically comprises volatile semiconductor random access memory (RAM). Each computer preferably includes a program product reader that accepts a program product storage device, from which the program product reader can read data (and to which it can optionally write data). The program product reader can comprise, for example, a disk drive, and the program product storage device can comprise removable storage media such as a magnetic floppy disk, an optical CD-ROM disc, a CD-R disc, a CD-RW disc, or a DVD data disc. In the embodiment were a plurality of computers are used, If desired, the computers can be connected so they can communicate with each other, and with other connected computers, over a network. Each computer can communicate with the other connected computers over the network through a network interface that enables communication over a connection between the network and the computer.

The computer operates under control of programming steps that are temporarily stored in the memory in accordance with conventional computer construction. When the programming steps are executed by the CPU, the pertinent system components perform their respective functions. Thus, the programming steps implement the functionality of the system as described above. The programming steps can be received from the DASD, through the program product reader, or through the network connection. The storage drive can receive a program product, read programming steps recorded thereon and transfer the programming steps into the memory for execution by the CPU. As noted above, the program product storage device can include any one of multiple removable media having recorded computer-readable instructions, including magnetic floppy disks and CD-ROM storage discs. Other suitable program product storage devices can include magnetic tape and semiconductor memory chips. In this way, the processing steps necessary for operation can be embodied on a program product. Alternatively, the program steps can be received into the operating memory over the network. In the network method, the computer receives data including program steps into the memory through the network interface after network communication has been established over the network connection by well-known methods that will be understood by those skilled in the art without further explanation.

Automated process control is known to those of skill in the art (for example, see U.S. Pat. Nos. 4,554,887; 5,576,946 and 6,629,003). Automated process control software is commercially available. For example, the software “Compu 4” from Vector Corporation (Marion, Iowa, USA) is a FDA 21 CFR Part 11 Ready Automated Process Control System that utilizes PC/PLC-based processors operating a Windows-based Supervisory Control and Data Acquisition (SCADA) platform. Operational control modes include manual and automated operation of the process. Real time process data is stored to generate batch reporting, trending and records.

The methods of manufacture herein can be used to prepare pharmaceutical compositions containing an agent that tends to associate into non-equilibrium structures, such as pre-micellar aggregates, macro-aggregates or micellar clusters or other structures that result in compositions with non-equilibrium surface tension. The methods of manufacture herein are applicable to the manufacture of pharmaceutical compositions described herein and any pharmaceutical compositions known in the art that contain an agent that tends to form non-equilibrium structures in liquid compositions. The methods produce pharmaceutical compositions that have a predetermined surface tension and that maintain such surface tension following manufacture, such as during subsequent packaging and storage.

During manufacture, shear flow and shear rate can influence the structure or distribution of components in a fluid. For example, rod-like micelles or aggregates of surfactants align their orientation with the direction of the flow field and generally orient in the shear direction (Schmidt et al., Rheo-optical investigations of lyotropic mesophases of polymeric surfactants, Rheol. Acta 38: 486-494 (1999). Under quiescent conditions, surfactant molecules in a fluid system containing low concentrations of surfactant generally exist as micelles and free monomers in a dynamic equilibrium. As the system is subjected to shear, the fluid in the system maintains its overall equilibrium as long as the relaxation rate is faster than the imposed shear rate. At higher shear rates, the micelles may be forced together by dynamic collisions, forming non-equilibrium structures. The dissociation of these non-equilibrium structures into stable micelles and free monomer in equilibrium may occur quickly or over a long period of time. For example, the dissociation of these non-equilibrium structures may take a fraction of a second or may take days or weeks or months to reach equilibrium. Turbulent flow in a fluid may exacerbate the formation of such non-equilibrium aggregates. Therefore, it is one objective herein to minimize the formation of non-equilibrium structures.

As described herein in, non-equilibrium aggregate formation can alter the surface tension of a composition during and after manufacture. Reduction of non-equilibrium aggregate formation can stabilize the surface tension of a composition. Aggregate formation, including non-equilibrium aggregate formation and modulation of surface tension characteristics of a composition, can be influenced by the manufacturing and storage processes. For instance, non-equilibrium aggregate formation can be induced by excessive energy input (e.g. vigorous mixing or agitation) in the manufacturing process. The methods of manufacture herein provide favorable conditions whereby non-equilibrium aggregate formation is reduced or eliminated, thereby stabilizing the surface tension of manufactured compositions.

1. Energy Input/Free Energy Decrease

In the methods herein, the energy input into the manufacturing processes is lowered or the free energy of the system is otherwise reduced. This results in a decrease in the amount of non-equilibrium aggregation of one or more agents in a composition. As discussed above, the general steps in a manufacturing process include, but are not limited to, mixing, transferring, and filtration of the compositions. Reduction of the free energy of the system or reduction of the applied energy can be effected at any one of these steps. Reduction of the free energy of the system or reduced applied energy input also can be effected at two or more steps, including at all of the steps. Reduction of the free energy of the system or reduction of the applied energy can be accomplished by a number of approaches that include, but are not limited to, reduction in applied energy of mixing and stirring, such as by reducing turbulence in mixing, reduction in applied pressure in the transfer of solutions thereby reducing the pressure differential, and reduction in the applied energy input in filtration steps.

a. Mixing

In one embodiment of a manufacturing process, an agent is mixed with one or more components of a composition in one or more mixing steps. The one or more mixing steps include mixing the agent and components or intermediates or the composition in a liquid form, such that the formation of non-equilibrium aggregates is reduced or eliminated. Mixing can include, but is not limited to, stirring, circulating, blending and rotating. The mixing is performed at reduced energy input such as by reducing the speed or power exerted during mixing. Reducing the energy input reduces the amount of non-equilibrium aggregates, including pre-micellar aggregates, formed in the composition, thereby forming a composition at equilibrium with a stable predetermined surface tension.

When fluids are stirred at slow speeds, for example in an unbaffled tank, the fluid remains in laminar flow and the liquid stays in flow lines. Such flow in a tank is known as circulation and is a low energy laminar flow mixing technique. For a low-viscosity liquid, a rotating impeller imparts tangential motion to the liquid. Without baffling, this swirling motion approximates solid-body rotation in which little mixing actually occurs. As the stirrer or impeller speed is increased, the liquid will move faster but will generally remain in flow lines. At higher rates of shear, the fluid near the impeller may experience transient flow or turbulent flow. As the energy input continues to increase, the fluid may be drawn into a vortex, which can incorporate air and draw suspended material into the impeller. The suspended matter can be damaged as a result of the collisions which occur between the impeller, liquid in turbulent flow, air and other suspended matter particles.

One method of increasing particle dispersion in a fluid in a tank using a lower energy input to the drive motor is the use of an angled or off-center impeller. The placement of the impeller off-center causes the flow lines of the fluid to collide with the tank wall, which introduces turbulent flow and mixing at the tank wall. This reduces, but does not eliminate swirl, and increases the dispersion of particulates throughout the fluid. Mechanical complications and the associated costs generally preclude the use of angled and off-center mounting with larger impellers.

Another method of increasing particle dispersion in a fluid in a tank using a lower energy input to the drive motor is to change the design of the impeller used. For example, replacing a flat-blade impeller with a pitched-blade impeller promotes axial flow that increases particle distribution in a fluid at the same energy input. The number of blades on the impeller, the number of impellers, the configuration and the placement of the blades of the impeller (for example, radial or axial, center of off-center or angled) all can determine the mass transfer energy of the impeller. For example, see Wu, J., Zhu, Y. & Pullum, L., “Impeller geometry effect on velocity and solids suspension,” Trans IChemE, 79(A), 989-997 (2001).

Another method of increasing particle dispersion in a fluid in a tank using a lower energy input to the drive motor is incorporating baffles into the tank design. The baffles can increase turbulent flow near the baffle leading edge, which at low energy levels can improve mixing. The presence of baffles produces axial flow, in which the discharge flow produced by the impeller impinges on the base of the container, flows radially to the container wall, then up the wall, returning to the impeller from above. This flow pattern can be inferred from the solids that are distributed rather uniformly throughout the liquid.

One purpose of baffling is to convert the swirling motion of a fluid mixed under lower energy into a preferred flow pattern to accomplish process objectives. The most common flow patterns are axial flow, typically used for blending and solids suspension, and radial flow, used for dispersion. However, baffling also has some other effects, such as suppressing vortex formation, increasing the power input and improving mechanical stability of the system. In some configurations, the baffles are placed such that there is a gap between the baffle and the tank wall. This placement of the baffle is referred to as an “off-set position.” An off-set baffle in a tank reduces the formation of dead zones near the baffle. A plurality of baffles can be placed into the tank to increase the amount of turbulent flow and mixing in the fluid.

The shape of the baffle also can be selected to impart the desired flow characteristics during agitation. Flat-plate baffles are normally selected because of their ease of manufacture and installation. Profiled baffles are often selected to eliminate stagnant regions in a system. Profiled baffles come in a number of different shapes, for example, triangular, semicircular, oblong, concave, convex and “beaver-tail.” Number, size, shape and placement of the baffles in the mixing tank can improve the mixing efficiency of the fluid at lower energy levels. For example, a system that uses four narrow flat-plate baffles having a width equal to approximately 2% of the container diameter that run the length of the container's straight side has very low drawdown power requirements, good particle distribution throughout the liquid, limited surface vortexing and good mechanical stability. One skilled in the art can choose the combination of baffle size, shape, number and placement to achieve process objectives, either from mechanical specifications and calculation or empirically. For example, Computational Fluid Dynamics (C.F.D.) is a fluid flow analysis engineering software tool which simulates many types of mixing applications. C.F.D. is used to predict mixing performance and for optimum impeller/tank/baffle selection and configuration.

In one example, energy input at one or more mixing steps is reduced by reducing the agitation of the mixture such that the presence of non-equilibrium aggregates is reduced or eliminated. Agitation can be reduced for example by minimizing the shear force applied during mixing, the speed of mixing and/or the amount of time of mixing. For instance, a mixing step can be carried out at low energy input to fully solubilize one or more agents in the solution such that they are monomeric in solution. Reducing agitation can include reducing aeration and foaming in the mixing step. In another example, the speed of mixing is reduced and the duration of mixing is increased such that the presence of non-equilibrium aggregates is reduced or eliminated. The reduced energy input at mixing results in the formation of a composition with a predetermined surface tension that is maintained subsequent to the completion of the mixing step. For example, an agent is mixed into a liquid, such as into an aqueous solution, to obtain a predetermined surface tension. The mixing is performed at reduced energy input, such as reducing the RPM of the impeller and concomitantly increasing the length of mixing time, whereby the agent forms monomers or other equilibrium structures such as micelles that are the predominant form of the agent in the composition. The mixing results in the formation of a composition that has achieved thermodynamic equilibrium and remains at equilibrium for an extended period of time, such that the surface tension is stable.

b. Product Transport

In another embodiment of a manufacturing process, the process includes one or more steps of transferring the composition or components or intermediates of the compositions. Transferring generally involves automated, non-manual transfer of liquid compositions through a conduit, such as a pipe or tube. Transferring can include, but is not limited to, transferring between different mixing steps, transferring between a step of mixing and a filtering step, transferring between a step of filtering and a step of filling, and transfer between storage containers, such as transferring between intermediate and final storage containers. Transferring can include the transfer of a composition to packaging such as by using blow-fill-seal technology.

One of the considerations that must be taken into account when sizing a system for large-scale manufacturing is the pressure differential or pressure drop. Pressure drop is proportional to the velocity and the viscosity of the fluid to be filtered. In most cases, pressure drop is proportional to the second power of velocity and to a power of less than 0.25 of viscosity. The calculation of pressure drop in a system can be simplified if the fluid is incompressible and is Newtonian. Most liquids are incompressible, which means that as equal flow rates the pressure differential will be independent of the absolute pressure of the system. A fluid is said to be a “Newtonian fluid” when the viscosity of the fluid is independent of the shear on the fluid. Many low-solids aqueous dispersions or solutions exhibit Newtonian flow. Addition of a polymer, such as xanthan gum, may change the rheology of the solution or dispersion so that it demonstrates non-Newtonian flow. For example, in the case of a solution that includes xanthan gum, the flow will become pseudoplastic, which means that the viscosity of the fluid depends on the shear that the fluid experiences. Calculation of pressure differential in a system with a non-Newtonian fluid is more complex than for Newtonian fluids but can be accomplished with formulae known to those skilled in the art.

Pressure differential or pressure drop losses in a system can be attributed to either laminar flow losses or turbulent flow losses. Laminar flow in tubes and piping generally occurs at Reynolds numbers less than 2,000. Turbulent flow in tubes and piping generally occurs at Reynolds numbers greater than 3,000. At Reynolds numbers between 2,000 and 3,000, the flow is unstable and may change from laminar flow to turbulent flow or vice versa. Turbulent losses in a system can occur due to the configuration of the system, including the pipes, fittings, filter housing, filter supports and sealing areas. At low flow rates, the laminar flow terms dominate and pressure drop is related to velocity. At higher flow rates, turbulent terms start to play a role, and turbulent flow at junctions, fittings and sealing areas increases as flow rate increases. Therefore, in scaled-up systems, decreasing the flow rate through the system reduces the pressure drop losses due to turbulent flow.

In the processes herein, one or more transferring steps in the manufacturing process are performed with reduced energy input, whereby the resulting composition maintains a pre-determined surface tension. Energy input is minimized such that compositions in equilibrium are maintained and non-equilibrium aggregates are not formed. Energy input can be reduced during transfer, for example, by reducing the pressure differential of the system and/or reducing the shear resulting from turbulent flow during transfer.

The pressure differential can be reduced by lowering the applied pressure that is used to transfer the solutions from one mixing tank to another container or a mixing tank to filtration, transferring for filling and/or transferring between storage containers. In one example, the pressure is reduced in transferring a composition between a mixing tank and an in-line filter. Pressure can be reduced, for example, by lowering the pressure applied to a conduit (e.g. tubes, pipes) used to transfer a composition, components or intermediates. Pressure can be lowered for example, by decreasing the flow rate of transfer, by increasing the diameter of the transfer conduit and by decreasing the length of the transfer conduit. In one example, the pressure used in the manufacturing system is lowered such that the pressure differential is reduced by 10×, 5×, 2×, 1.9×, 1.8×, 1.7×, 1.5× 1.4×. 1.3×, 1.2×, 1.1×. For example, the pressure differential can be reduced by 10, 9, 8, 7, 6, 5, 4, 3, 2 or 1 psig. In one example, the pressure differential for transferring is reduced two-fold, from 10 psig to 5 psig.

c. Filtration

In another embodiment of a manufacturing process, the process includes one or more steps of filtering the composition or components or intermediates of the compositions. The filtration is performed with reduced energy input such that a composition in equilibrium is maintained and non-equilibrium aggregates are not formed, such that the composition maintains a predetermined surface tension.

Filtration processes may directly effect yield, product consistency and reproducibility of pharmaceutical products. Process parameters to be considered include type of filtration used, batch size, temperature, time, pressure differential, and flux (flow per unit area). In one embodiment of a manufacturing process, membrane filtration is used.

Membrane filtration is a process that separates particles from the fluid using size exclusion by providing a barrier to particles larger than the pore-size rating of the membrane. The membrane pore size generally can range from about 0.1 μm to greater than 10 μm, and generally a membrane pore size of less than 0.65 μm is used to remove whole cells, cell debris and other particulates. The tight membranes in the 0.1-0.22 μm-range are generally used for filter sterilization. The membrane separates particles based on different sizes and shapes of the particles and the pores of the membrane. Particles larger than the size of the membrane's pores are retained by the membrane, while smaller particles pass through. Besides the pore size, the electric charge of the membrane surface and the diffusivity of the separated particles may play a role.

Filter performance under normal flow conditions is determined by a number of factors, including capacity. Capacity is the volume of fluid that can be processed at a maximum differential pressure. Generally, the higher the flow rate, the lower the capacity. Capacity can be determined empirically. Capacity measurements can be done using constant flow (a maximum pressure limit) or constant pressure (a minimum flow limit). The use of prefilters to reduce the suspended solids in the fluid prior to its reaching a tight membrane, such as a 0.22 μm, 0.2 μm or 0.1 μm sterilizing filter will increase the process capability of the membrane filter by removing larger particulates that would foul the sterilizing filter from the fluid stream prior to contact with the sterilizing filter. Prefilters can include lenticular depth filters such as cellulosic pad filters, single- and/or dual-layer high retention media, and single- and/or dual-layer high charge media, pleated prefilters, such as glass microfiber, polypropylene or membrane, and a wrapped depth filter.

In general membrane filtration systems, the application of a pressure differential is the driving force for separation of particles from the fluid. For microfiltration applications using membranes, the pressure differential is a hydrostatic pressure difference. Rate of flow in a membrane filtration process is the product of the membrane area and the applied differential, and all other factors being equal, the pore size of the membrane in the process usually determines the flow rates (Jornitz M. W. and T. H. Meltzer, Sterile Filtration: A Practical Approach, Marcel Dekker, New York, Chapter 4, Flow and Pressure, 1991). Other factors that influence the rates of flow include filter design, pore shape and length, pore distribution and total porosity of the membrane.

In one embodiment, the filtration step in the disclosed method is performed at reduced energy input levels. For example, energy input is reduced by decreasing the flow rate versus pressure differential at one or more filtration steps. The pressure differential before and after the filter can be adjusted such that equilibrium structures of an agent are maintained, for example an agent is maintained as a monomer or in equilibrium micelles. The filtration steps is performed such that non-equilibrium structures are not formed.

As discussed above, pressure differential or pressure drop must be taken into consideration when sizing a system for large-scale manufacturing, and this includes sizing any filtration system incorporated into the system. The maximum allowable pressure drop will control the minimum membrane area needed to do the job. Because the characteristic flow length for membrane filters is small, the liquid flow through the membrane is generally laminar flow. Turbulent losses in a filter system can occur due to the configuration of the filter housing, filter supports, pipes, and sealing areas. At low flow rates, the laminar flow terms dominate and pressure drop is related to velocity. At higher flow rates, turbulent terms start to play a role. For the membrane alone, pressure drop is proportional to viscosity. For the filter housing alone, pressure drop is independent of viscosity of the fluid but is dependent on flow rates, indicating turbulent losses. Therefore, in scaled-up filtration systems, which may include integrated filter and filter housing combinations, pressure differential or drop is altered by filter and housing losses.

Several filtration systems, including membrane systems that use flat sheets, hollow fiber and spiral wound modes, are known to the skilled artisan. Theoretical aspects of membrane processes have been discussed by Fane and Radovich (Fane, A. G. and Radovich, J. M., “Membrane Systems,” in Separation Process in Biotechnology, ed. by J. A. Asenjo, Marcel Dekkar, New York, 1990) and others.

2. Surface Tension

In the manufacturing processes herein, the surface tension of the compositions is maintained at a pre-determined range. The pre-determined range will depend on the desired surface tension of the manufactured compositions. For example, a desired surface tension can be chosen that enhances the delivery, effectiveness or maintenance of an effect of a pharmaceutical composition. In one embodiment, the predetermined surface tension is chosen to effect deposition, penetration and/or retention of the composition or an agent contained in the composition upon administration of the composition. In one example, the predetermined surface tension of the compositions is between 10 dynes/cm and about 70 dynes/cm. In another example, the predetermined surface tension of the compositions is between 20 dynes/cm and about 60 dynes/cm. In another example, the predetermined surface tension of the compositions is between 30 dynes/cm and about 50 dynes/cm. In yet another example, the predetermined surface tension of the compositions is between 35 dynes/cm and about 45 dynes/cm.

The methods herein can be used to manufacture pharmaceutical compositions such that the predetermined surface tension is maintained at the predetermined surface tension or range of surface tension subsequent to manufacture. Maintaining a surface tension refers to minimal variation in the surface tension of the composition from the predetermined range. For example, the processes herein can result in a variation of surface tension of less than 50%, 45%, 40%, 35%, 30%, 25%, 20%, 15%, 10%, 8%, 6%, and 5% as compared between the predetermined surface tension or predetermined surface tension range and the surface tension measured subsequent to the manufacturing process in the manufactured composition.

Maintenance of surface tension also can be assessed by comparing the surface tension after manufacture of a composition to surface tension of the composition following storage. For example, surface tension can be assessed directly after manufacture, including surface tension measurements of the final solution in batch and of the final solution after a filling step, such as after a filling step into single-dose or multi-dose containers or containers. Surface tension can then be assessed after storage, including hours, days, weeks, months and years after manufacture. For example, surface tension of individual samples can be assessed after a period of storage. In the methods herein, surface tension of a manufactured pharmaceutical composition is maintained during storage such that the surface tension of the composition varies less than 50%, 45%, 40%, 35%, 30%, 25%, 20%, 15%, 10%, 8%, 6%, and 5%, as compared between the surface tension directly after manufacture (e.g. just subsequent to the filling step) and the surface tension after storage of the composition.

Surface tension can be measured by using a ring tensiometer or the capillary rise measure method, which includde a capillary tube of known diameter placed into the liquid and a measurement of capillary rise taken to provide surface tension. Surface tension also can be measured by the spinning drop method, pendant drop method, bubble pressure method, drop volume method, and Wilhelmy plate method.

3. Manufacture of Compositions with Low Concentrations of Surfactant

In an example of the methods herein, compositions containing a surfactant at a concentration at or near the cmc of the surfactant and one or more agents that form non-equilibrium aggregates are manufactured with a predetermined surface tension. The surfactant concentration is adjusted to be at or near the cmc such that the composition has a predetermined surface tension. Additionally, the amount of surfactant is chosen to reduce side effects of the composition when administered.

The methods of manufacture herein reduce the formation of non-equilibrium aggregates of an agent contained in the composition such that the aggregates do not alter the surface tension from the predetermined surface tension. In compositions containing low amounts of surfactants, the number of molecules contributing to surface activity is reduced as compared to compositions with saturating amounts of surfactant (i.e. concentrations above the cmc of the surfactant). Consequently, addition of an agent that has surface activity and/or indirectly affects surface tension can alter the surface tension of a composition containing a concentration of surfactant below the cmc. If the composition contains an agent that tends to form non-equilibrium aggregates, surface tension of the composition can be unstable to particular conditions of manufacture. The methods of manufacture provide conditions favorable to the production of such compositions with a stable predetermined surface tension.

In an exemplary embodiment, an aqueous composition is formulated containing an agent that forms non-equilibrium aggregates, for example tobramycin, and a surfactant, for example a non-ionic surfactant such as Tween®-20, at a concentration near the cmc. Tobramycin can form non-equilibrium aggregates in aqueous compositions that can alter the surface tension of the composition. The methods of manufacture of tobramycin-low surfactant compositions includes conditions where non-equilibrium aggregates are not formed or are not maintained. The methods include reducing the energy input in one or more steps of manufacture, including mixing, transferring and filtering. For example, the pressure differential during transfer of the composition is reduced. Additionally, the pressure exerted on the composition during filtration also is reduced. The manufactured composition contains tobramycin in equilibrium structures in the aqueous composition, e.g. partitioned into stable mixed micelles or as free monomers. The manufactured composition has a predetermined surface tension that is maintained following manufacture. In one example, the predetermined surface tension is between about 30 and 50 dynes/cm. In another example, the predetermined surface tension is between about 35 and 45 dynes/cm. The predetermined surface tension remains stable through storage.

F. Articles of Manufacture and Kits

The compositions disclosed herein may be packaged as articles of manufacture including packaging material, a composition disclosed herein, which is effective for treatment, prevention or amelioration of one or more symptoms of a disease or a disorder, within the packaging material, and a label that indicates that the composition is used for treatment, prevention or amelioration of one or more symptoms of a disease or a disorder.

The articles of manufacture provided herein contain packaging materials. Packaging materials for use in packaging pharmaceutical products are well known to those of skill in the art. See, e.g., U.S. Pat. Nos. 5,323,907; 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the compounds and compositions provided herein are contemplated as are a variety of treatments for any disease or disorder. The diseases or disorders for which the article of manufacture is useful include, but are not limited to, diseases and conditions of infection and inflammation, such as acute, recurrent and chronic sinusitis.

In one embodiment, an article of manufacture includes a composition at a predetermined surface tension aseptically packaged into containers or containers. The articles of manufacture can include any of the compositions described herein. Typically, such articles of manufacture are packaged in a closed system under sterile conditions. The containers are filled and then hermetically sealed shut, such as by heat treatment. In one such example, compositions are filled and sealed using the Blow-Fill Seal method (see for example, Business Briefing Pharmagenerics 2002). The articles of manufacture can include any containers that can be filled under sterile conditions and hermetically sealed. Containers include any containers suitable for pharmaceutical compositions known in the art including, but not limited to, capsules, ampoules, bottles, vials, bag, blister, and cartridges. Such containers can be any suitable materials known in the art including, but not limited to, glass, borosilicate glass and plastics, such as polyethylene and polypropylene. Such containers are aseptically and hermetically sealed using methods known in the art. Such seals include, but are not limited to heat, sealing, vacuum sealing, snap cap, safety-seal cap, heat-sealed cap, sealed and syringe. In one example, the article of manufacture includes a container made and sealed using blow-fill-seal technology. Blow-fill-seal (BFS) describes an aseptic filling process in which hollow containers are blow molded, filled with sterile product, and sealed, all in one continuous machine cycle.

Any one of the compositions disclosed herein may be supplied in a kit along with instructions on conducting any of the methods disclosed herein. Instructions may be in any tangible form, such as printed paper, a computer disk that instructs a person how to conduct the method, a video cassette or digital video device containing instructions on how to conduct the method, or computer memory that receives data from a remote location and illustrates or otherwise provides the instructions to a person (such as over the Internet). A person may be instructed in how to use the kit using any of the instructions above or by receiving instructions in a classroom or in the course of treating a patient using any of the methods disclosed herein.

In one example, a kit includes any one of the compositions herein for use in the treatment of sinusitis and instructions for use. Instructions for use can include instructions for nebulization, such that the composition is administered as an aerosol. A kit also can include a nebulizing device. Any nebulizing device capable of forming respirable particles (e.g, particles having a mass median aerodynamic diameter (MMAD) of about 1.0 to 6.0 microns) from a composition as described herein may used, including both jet nebulizers and ultrasonic nebulizers. Such nebulizing devices are known in the art. For example, such devices are described in U.S. Pat. Nos. 4,094,317; 4,805,609; 4,113,809; 4,832,012; 4,961,885; 5,049,388; 5,170,782; 5,277,175; 5,666,946; and 6,152,383. An example jet nebulizer is described in U.S. Pat. No. 4,832,012. Exemplary jet nebulizing devices include the Medicaid Sidestrearn jet nebulizer and the Pari LC jet nebulizer from PAR1 Respiratory Equipment, Richmond, Va., USA. An example ultrasonic nebulizer is described in U.S. Pat. No. 5,170,782. Exemplary ultrasonic nebulizing devices include the Ultraneb 99, the Porta-Sonic™ and the Pulmo-Sonic™ nebulizers produced by The DeVilbiss Co., Somerset, Pa., USA.

Compositions and kits and articles of manufacture containing such compositions can include single dose or multi-doses of a composition. Compositions can be for direct administration or dilution. Suitable diluents include water, saline, alcohol and other any diluents known in the art for diluting pharmaceutical compositions. Unit doses can include compositions prepared in a volume between about 0.5 and 6.0 mls, including, but not limited to between about 2 and 4 mls and about 2.5 and 3.5 mls. For example, such volumes can include 0.5, 1, 1.5, 2, 2.5, 3, 3.5, 4, 4.5, 5, 5.5. and 6 ml.

### G. EXAMPLES

The following examples are included for illustrative purposes only and are not intended to limit the scope of the invention.

### Example 1

### Small Scale Formulation of Pharmaceutical Compositions

A. Formulation

Compositions were prepared containing antibiotics as agents for treatment of sinusitis. Surfactant was added to the compositions to adjust the surface tension. A stock solution of surfactant was prepared by mixing 10 ml of Tween-20 (Spectrum) and 990 ml sterile water (sterile water for irrigation; Abbott, Abbott Park, Ill.). Antibiotics were formulated with the surfactant solution as follows:

Gentamicin: 107.3 g (approximately 37.8 ml powder volume) gentamicin sulfate powder (Spectrum) was mixed with 23.8 ml surfactant solution. Sterile water (Abbott) and sterile saline were added in equal proportions, 1019.2 ml each, to a final volume of 2100 ml. The pH of the solution was confirmed to be between 4 and 6.9. The solution was filtered through a 0.22 micron sterile filter and transferred by appropriate volume aliquots into unit of use vials. Each vial received a single 3 ml dose of the formulation containing 95 mg of gentamicin.

Ciprofloxacin: 4500 mg of ciprofloxacin tabs (Bayer) were ground to a fine powder with a mortar and pestle. 1.98 ml of surfactant solution was added and the mixture was further ground with a mortar and pestle. Sterile water (172.42 ml) was added and the solution was stirred until completely dissolved. 393.75 mg of NaCl powder was added and dissolved into the solution. The pH was adjusted to between 4.0 and 6.9. The solution was filtered through a 0.22 micron sterile filter and transferred by appropriate volume aliquots into unit of use vials. Each vial received a single 3.5 ml dose of the formulation containing 90 mg of ciprofloxacin. A second formulation of ciprofloxacin was compounded following the above method using ciprofloxacin powder in place of ciprofloxacin tabs.

Ceftazidime: Small batches were formulated in separate vials. Each vial containing 6 g (approximately 5 ml powder volume) of Fortaz® (GlaxoSmithKline) was reconstituted with 27.36 ml water (Baxter, Deerfield, Ill.) and mixed with 0.37 ml surfactant solution. The pH of the solution was adjusted to between 4.0 and 6.9, as necessary. The solution was filtered through a 0.22 micron sterile filter and transferred by appropriate volume aliquots into unit of use vials. Each vial received a single 3 ml dose of the formulation containing 550 mg of cefatazidime.

Levofloxacin: 123.2 ml of levofloxacin solution at a stock concentration of 25 mg/ml (Ortho-McNeil, Raritan, N.J.) was mixed with 7.3 ml sterile water (Baxter) and 1.5 ml surfactant solution. The pH of the solution was confirmed to be between 4 and 6.9. The solution was filtered through a 0.22 micron sterile filter and transferred by appropriate dose volume aliquots into unit of use vials. Each vial received a single 3 ml dose of the formulation containing 70 mg of levofloxacin. A second formulation of levofloxacin was compounded following the above method using levofloxacin tablets in place of levofloxacin solution.

Ofloxacin was formulated in sterile water and surfactant solution using a similar formulation to levofloxacin. The pH of the solution was confirmed to be between 4 and 6.9. The solution was filtered through a 0.22 micron sterile filter and transferred by appropriate dose volume aliquots into unit of use vials. Each vial received a single 3 ml dose of the formulation containing 90 mg of oxofloxacin.

Tobramycin: 90.7 g (approximately 19.6 ml powder volume) of tobramycin powder (Spectrum) was mixed with 1283.7 ml of sterile water (Abbott) and 1283.7 ml of sterile saline. 30.6 ml of surfactant solution was then added and mixed. The pH of the solution was adjusted with citric acid solution to a pH between 4.0 to 6.9. The solution was filtered through a 0.22 micron sterile filter and transferred by appropriate volume aliquots into unit of use vials. Each vial received a single 3 ml dose of the formulation containing 95 mg of tobramycin. Formulations of tobramycin starting with either tobramycin SO₄ powder and tobramycin base powder were compounded following the above method.

In the formulations described, each composition was manually filtered by pushing the solutions through a 0.22 micron sterile filter from an attached syringe. The solutions were filled into single dose vials using a syringe or using a Wheaton pump into clean vials that were sealed manually.

B. Physical Properties

Physical properties of antibiotic compositions formulated with surfactant were measured, including pH, osmolality and surface tension.

1. pH Measurement

pH was measured using a Denver Instrument UB-5 pH meter. The instrument was calibrated with a two point calibration method using fresh pH 7.00 and pH 4.00 buffer solutions. Before each measurement, the probe was washed clean with Millipore 18 Ω water, followed by blotting dry with a Kimwipe. Solutions were allowed to stabilize (>30 minutes) at room temperature (25±0.3 C) prior to pH measurement. The solution was briefly stirred with the probe, then allowed to stabilize for >1 minute, at which point the pH was recorded. After the pH was recorded, the probe was rewashed with Millipore 18 Ω water, blotted dry, and placed back in pH 7.00 buffer to verify that the calibration was still accurate. Results are shown in Table 1.

2. Osmolality

A Vapro® Vapor Pressure Osmometer from WESCOR was used to measure osmolality. The osmometer was calibrated in triplicate using a three point calibration at 100, 290, and 1000 mOsm/kg H₂O. These standards were purchased from Fisher. A clean test was run on the osmometer after the calibrations were complete to measure stability and yielded a negligible contamination level of 0 (cleaning of the thermocouple head is required when a contamination level of 10 is reached). Each sample measurement was done in triplicate for accuracy, and an empty run was conducted between different samples to prevent cross contamination. The calibration standard at 290 mmol/Kg was run every 5 samples and found to be stable throughout the measurements. Osmolality measurements are shown in Table 1.

3. Surface Tension

The surface tension was measured using a Nima ST9000 tensiometer, applying the Wilhemly plate technique. The tensiometer was calibrated at 48.3 mN/m using the supplied calibration weight. New Wilhelmy plates were used for each sample, and were washed thoroughly with water before use to remove contaminants. Once the new plate was washed, the plate was lowered into solution (equilibrated for >30 minutes at 25±0.3 C) and allowed to equilibrate. The plate was then raised from solution and zeroed. A successive number of 8 measurements was taken and averaged for each solution. Appropriate rezeroing was done between measurements. The same solution was then remeasured eight times with a new Wilhelmy plate to average any inconsistencies between Wilhemly plates. Surface tension results are shown in Table 1.

TABLE 1

| | | Surface tension | Osmolality |
|---|---|---|---|
| | pH | (mN/m @ 25° C.) | (mOsm/kg H₂0) |
| Ceftazidime | 6.79 | 29.8 | 727.0 |
| Ciprofloxacin Tabs | 4.76 | 41.3 | 125.2 |
| Ciprofloxacin Powder | 4.33 | 38.7 | 141.4 |
| Gentamicin Sulfate | 4.54 | 39.1 | 208.9 |
| Levofloxacin Injection | 4.88 | 37.3 | 79.2 |
| Levofloxacin Tablets | 7.28 | 39.2 | 310.2 |
| Tobramycin SO₄ Powder | 7.45 | 39.3 | 220.9 |
| Tobramycin Base | 6.52 | 38.8 | 161.7 |
| Powder | | | |
| Pure Millipore Water | | 73.0 | |

The surface tension of the compositions was between about 30 to about 50 mN/m.

### Example 2

### Manufacture of Tobramycin Compositions Under Conditions that do not Maintain a Predetermined Surface Tension

A 20 L batch of Tobramycin Solution was manufactured as follows. Approximately 60% of the final product weight of Water for Injection (WFI) was transferred from a loop, cooled in a jacketed tank to 77-86° F., and transferred to a formulation tank. Nitrogen that was 0.2 μm filtered was supplied through a dip tube to the formulation tank to sparge the solution during mixing to minimize the potential for degradation of the tobramycin. The product tank mixer was activated using a dial setting of 2 and sodium chloride was added to the tank and mixed for two minutes.

Polysorbate 20 was added to the tank and the solution was mixed for 4 minutes. 10 N sulfuric acid was added to the tank and the solution was mixed for 5 minutes. The solution was mixed for an additional 35 minutes and allowed to cool to room temperature. The pH was adjusted with two small aliquots of 10 N H₂SO₄ with 13 minutes of mixing between each of the additions. Tobramycin base was added to the tank and the solution was mixed for 50 minutes. The pH was adjusted to 6.0 using 5 aliquots of 10 N sulfuric acid with 10 minutes of mixing between each of the additions. The solution was brought to the final weight with WFI and mixed for 15 minutes.

A single new 0.1 micron Pall N66 STW-SLK7002NTP filter was installed. The blow fill seal (BFS) machine product path, sterilizing filter, and fill system were steam sterilized-in-place. The Tobramycin Solution was aseptically filled into 3 mL disposable plastic unit dose ampoules using blow-fill seal (BFS) technology. The pressure used to filter the solution was not measured, but was estimated to be at least 10 psig. The BFS machine forms an ampoule, aseptically fills each ampoule with product solution, and hermetically seals the ampoule in one continuous motion. Complete cards of ampoules were conveyed out of the machine for packaging and testing.

Pre-filtration, post-filtration prior to filling, and post-filling samples were collected and tested for surface tension. The results are shown in Table 2. In addition, post-filling samples were tested for surface tension during storage as shown in Table 3.

TABLE 2
Results Batch 02103B

| Sample | Surface Tension (mN/m) |
|---|---|
| Pre-filtration | 38 |
| Post-filtration; pre-filling | 57 |
| Post-filling | 55 |

The results demonstrated that the filtration process resulted in a surface tension that fell outside the range of the predetermined surface tension of 30 to 50 nM/m for the composition.

TABLE 3

| | | Surface tension |
|---|---|---|
| Storage temperature | Time point sampled | (dynes/cm) |
| 5° C. | Initial | 55 |
| | 1 month | 56 |
| | 3 months | 50 |
| | 4 months | 41 |
| | 6 months | 47 |
| 25° C. | 1 month | 51 |
| | 3 month | 41 |
| | 6 months | 44 |

### Example 3

### Comparison of Parameters for Manufacture of Tobramycin Compositions

A 20 L batch of Tobramycin Solution was manufactured as follows. Approximately 60% of the final product weight of Water for Injection (WFI) was transferred from a loop, cooled in a jacketed tank to 77-86° F., and transferred to a formulation tank. Nitrogen that was 0.2 μm filtered was supplied through a dip tube to the formulation tank to sparge the solution during mixing to minimize the potential for degradation of the tobramycin. The product tank mixer was activated using a dial setting of 2 and sodium chloride was added to the tank and mixed for 2 minutes. Polysorbate 20 was added to the tank and the solution was mixed for 7 minutes. 10 N sulfuric acid was added to the tank and the solution was mixed for 2 minutes. The solution was mixed for an additional 61 minutes and allowed to cool to room temperature.

Tobramycin base was added to the tank and the solution was mixed for 14 minutes. The pH was adjusted to 6.1 using 3 aliquots of 10 N sulfuric acid with approximately 10 minutes of mixing between each of the additions. The solution was brought to the final weight with WFI and mixed for 15 minutes. The 20 L batch of solution was divided into four 5 L sub-batches and each sub-batch was exposed to different conditions. A description of the processing parameters and surface tension results for each sub-batch is provided below.

Sub-Batch 05403B

5 L of Tobramycin Solution was filtered through a 0.1 micron Pall N66 STW-SLK7002NTP filter into a glass bottle using approximately 10 psig of pressure applied to the tank. Surface tension results for a sample of the filtered solution was measured and found to be 57.6 mN/m.

Sub-Batch 05403C

5 L of Tobramycin Solution was filtered through a 0.1 micron Pall N66 STW-SLK7002NTP filter into a glass bottle with approximately 5 psig of pressure applied to the tank. Surface tension results for a sample of the filtered solution was measured and found to be 44.4 mN/m.

Sub-Batch 05403D

Polysorbate 20 was added to 5 L of solution to adjust the concentration to five times the amount used in the batch. The solution was filtered through a 0.1 micron Pall N66 STW-SLK7002NTP filter into a glass bottle with approximately 10 psig of pressure applied to the tank. Surface tension results for a sample of the filtered solution was measured and found to be 37.4 mN/m.

Sub-Batch 05403E

5 L of Tobramycin Solution was filtered through a 0.1 micron Millipore LAVL04TP6 filter into a glass bottle with approximately 10 psig of pressure applied to the tank. Surface tension results for a sample of the filtered solution was measured and found to be 39.8 mN/m.

The results indicated that a predetermined surface tension of about 30 mN/m to about 50 nM/m, in particular of about 35 mN/m to about 45 nM/m could be achieved under condition including a reduction in filtration pressure, an increased surfactant concentration, and the use of less tightly wound filter. The results are summarized in Table 4.

TABLE 4
Comparison of Surface tension post-filling

| Batch | Conditions altered | Surface tension (mN/m) |
|---|---|---|
| 0504B | Control | 57.6 |
| 0504C | Reduced pressure and | 44.4 |
| | flow rate | |
| 0504D | Increased surfactant | 37.4 |
| 0504E | filter | 37.9 |

### Example 4

### Comparison of Filtration Parameters for Manufacture of Tobramycin Compositions

A 1 L batch of Tobramycin Solution was manufactured as follows. Approximately 60% of the final product weight of Water for Injection (WFI) was transferred to a formulation container. Sodium chloride and polysorbate 20 were added to the container and the solution was mixed. The pH was adjusted with aliquots of 10 N H₂SO₄ and the solution was mixed. Tobramycin base was added to the tank and the solution was mixed. The pH was adjusted with 10 N sulfuric acid and/or with 1 N sodium hydroxide to pH 5.8-6.2. The solution was brought to the final weight with WFI and mixed for 15 minutes. About 150 mL Tobramycin Solution was transferred to a pre-sterilized 400 mL beaker. The solution was filtered through one of the disk filters listed in Table 5. Platinum-cured silicon tubing (by Cole Parmer, ID; 0.250″, OD: 0.483″, Wall: 0.094″) was used together with a peristaltic pump. The filtered solution was collected in a glass beaker and transferred to glass vials for surface tension analysis. The results are presented in Table 5.

TABLE 5
Surface Tension Results

| | Surface |
|---|---|
| Filter | Tension Mean (mN/m) |
| Prefilter (control) | 39.5 |
| Bio Inert (0.1 μm, by Pall, part# FTKNTL) | 38.4 |
| Millipak-20, Gamma Gold (0.22 μm by | 38.4 |
| Millipore, part# MPGL02GH2) | |
| AcroPak-20 (0.1 μm by Pall, part# 2208) | 38.4 |
| Supor 47 mm (0.1 μm by Pall, part# | 41.1 |
| SCS100) | |
| Ultipor N66 (0.1 μm by Pall, part# FTK-NT) | 41.9 |

The results demonstrated that the increase of surface tension observed in Tobramycin Solution Lot 02103B (Example 2) was not due to the sorption of the surfactant (Polysorbate 20) used in the formulation by the filter. Results (Table 5) also indicated that use of disk filters with five different types of membranes resulted in the production of Tobramycin Solution with a surface tension results in the desired (predetermined) range of about 30 to about 50 dynes/cm.

### Example 5

### Manufacture of Tobramycin Compositions Under Conditions that Reduce Non-Equilibrium Aggregate Formation

A 20 L batch of Tobramycin Solution (batch 06003A) was manufactured as follows. Approximately 60% of the final product weight of Water for Injection (WFI) was transferred from a loop, cooled in a jacketed tank to 77-86° F., and transferred to a formulation tank. Nitrogen that was 0.2 μm filtered was supplied through a dip tube to the formulation tank to sparge the solution during mixing to minimize the potential for degradation of the tobramycin. The product tank mixer was activated using a dial setting of 2 and sodium chloride was added to the tank and mixed for 2 minutes.

Polysorbate 20 was added to the tank and the solution was mixed for 6 minutes. 10 N sulfuric acid was added to the tank and the solution was mixed for 3 minutes. The solution was mixed for an additional 15 minutes and allowed to cool to room temperature. Tobramycin base was added to the tank and the solution was mixed for 7 minutes. The pH was adjusted to 5.9 using 4 aliquots of 10N sulfuric acid with mixing between each of the additions. The solution was brought to the final weight with WFI and mixed for 27 minutes.

A single new 0.1 micron Millipore LAVL04TP6 filter was installed. The blow fill seal (BFS) machine product path, sterilizing filter, and fill system were steam sterilized-in-place. The Tobramycin Solution was aseptically filled into 3 mL disposable plastic unit dose ampoules using BFS technology. The pressure used to filter the solution was 5 psig. Complete cards of ampoules were conveyed out of the machine packaging and testing. Finished samples were taken and tested for surface tension and found to be 42.8 mN/m.

Results (Table 6) demonstrated that the use of the Millipore filter and the reduction in filtration pressure resulted in the production of Tobramycin compositions with surface tension that fell into the predetermined surface tension range. Additional compositions of tobramycin with a predetermined surface tension are provided in Table 7.

TABLE 6

| | | Tobramycin | |
|---|---|---|---|
| | | concentration | Surface tension |
| Batch | Volume | (mg/ml) | (nM/m) |
| 02103B | 20 L | 66.7 | 54.8 |
| 0504E | 5 L | 66.7 | 37.9 |
| 06003A | 20 L | 60 | 42.8 |

TABLE 7
Additional Compositions of Tobramycin with a predetermined surface
tension

| Ingredients | Formulation-1 | Formulation-2 | Formulation-3 |
|---|---|---|---|
| Tobramycin base | 60 mg/ml | 30 mg/ml | 15 mg/ml |
| Polysorbate 20 | 0.125 mg/ml | 0.125 mg/ml | 0.125 mg/ml |
| Sodium Chloride | 2.5 mg/ml | 3.6 mg/ml | 4.3 mg/ml |
| Sulfuric Acid | pH adjustment | pH adjustment | pH adjustment |
| Sodium | pH adjustment | pH adjustment | pH adjustment |
| Hydroxide | | | |
| Water for | QS | QS | QS |
| Injection | | | |

### Example 6

### Exemplary Pharmaceutical Compositions Formulated with Low Concentrations of Surfactant

Exemplary formulations with a predetermined surface tension of about 30 to about 50 nM/m are manufactured using the methods herein. In one example, the method set forth in Example 5 is used to manufacture the formulations listed in Table 8. Each manufactured composition contains a non-ionic surfactant, such as Tween®-20, at a concentration near the critical micelle concentration. For exemplary purposes, the concentration of Tween®-20 is 0.1 mM (0.01% by weight). Following manufacture, the surface tension is measured using the Wilhemly plate technique as described herein. The dosages of agents are listed in Table 8. The manufactured compositions are filled into single dose vials, for example, using blow-fill-seal technology, with a volume of 3 ml/vial.

TABLE 8

| | Exemplary |
|---|---|
| | Dosage |
| Agent | Range (mg) |
| Azithromycin | 50-400 |
| Acetylcysteine | 125-500 |
| Amikacin | 50-500 |
| Amphotericin B | 2.5-45 |
| Azelastine | 137-1096 mcg |
| Aztreonam | 250-1000 |
| Beclamethasone | 0.1-4 |
| Betamethasone | 0.1-4 |
| Cefazolin | 250-1000 |
| Cefepime | 125-1000 |
| Cefonicid | 250-1000 |
| Cefoperazone | 250-1000 |
| Cefotaxime | 250-1000 |
| Cefotetan | 250-1000 |
| Cefoxitin | 250-1000 |
| Ceftazidime | 250-1000 |
| Ceftizoxime | 250-1000 |
| Ceftriaxone | 250-1000 |
| Cefuroxime | 100-600 |
| Cephapirin | 250-1000 |
| Cipofloxacin | 25-200 |
| Clindamycin | 50-600 |
| Dexamethasone | 0.1-4 |
| Doxycycline | 10-100 |
| Erythromycin | 50-600 |
| lactobionate | |
| Fluconazole | 12.5-150 |
| Flunisolide | 0.1-4 |
| Flurbiprophen | 0.01-2 |
| Fluticasone | 10-700 mcg |
| Gentamicin | 10-200 |
| Ibuprofen | 25-400 |
| Itraconazole | 12.5-150 |
| Ketoralac | 0.05-4 |
| Levofloxacin | 40-200 |
| Linezolid | 50-600 |
| Loratidine | 0.5-10 |
| Mezlocillin | 300-1500 |
| Miconazole | 12.5-300 |
| Montelukast | 0.5-15 |
| Mupiricin | 1-25 |
| Nafcillin | 250-1000 |
| Ofloxacin | 25-200 |
| Oxacillin | 250-1000 |
| Oxymetazoline | 0.05-5 |
| Phenylepherine | 5-50 |
| Piperacillin | 100-1000 |
| Rifampin | 500-5000 |
| Taurolin | 5-200 |
| Tetrahydrozolidine | 0.05-5 |
| Ticarcillin/clavulanic | 500-5000 |
| Tobramycin | 10-200 |
| Triamcinalone | 0.05-3 |
| Vancomycin | 50-400 |
| Xylometazoline | 0.05-4 |
| Zafrilukast | 2-60 |

Since modifications will be apparent to those of skill in this art, it is intended that this invention be limited only by the scope of the appended claims.

1. A method of manufacturing a composition with a predetermined surface tension, comprising:
formulating at a manufacturing scale and under conditions that reduce or inhibit the formation of non-equilibrium aggregates, a pharmaceutical composition that contains an agent, whereby the composition has a predetermined surface tension.

2. The method of claim 1, wherein:
the composition comprises one or more surfactants; and
the concentration of the surfactant is near the critical micelle concentration (cmc).

3. The method of claim 1, wherein the composition comprises the agent in monomeric form.

4. A method of manufacturing a composition, comprising:
formulating at a manufacturing scale a pharmaceutical composition that has a predetermined surface tension, wherein:
the composition comprises an agent and a surfactant; and
the concentration of the surfactant is near the critical micelle concentration, whereby the manufactured composition has a predetermined surface tension.

5. The method of claim 4, wherein the concentration of the surfactant is below the critical micelle concentration.

6. The method of claim 4, wherein the concentration of the surfactant is about or equal to the critical micelle concentration.

7. The method of claim 4, wherein the concentration of the surfactant is within 2 fold or 3 fold below or above the critical micelle concentration.

8. The method of claim 4, wherein the predetermined surface tension is between 30-50 dynes/cm.

9. The method of claim 8, wherein the predetermined surface tension is between 35-45 dynes/cm.

10. The method of claim 4, wherein the agent is selected from the group consisting of an anti-infective agent, an anti-inflammatory agent, an antihistamine, an antileukotriene, a decongestant, an antiviral agent and an anesthetic.

11. The method of claim 10, wherein the anti-infective is selected from the group consisting of an aminoglycoside, a macrolide, a penicillin, a quinolone, a cephalosporin, an amphotericin B, a polyene, a pyrimidine analog and an azole agent.

12. The method of claim 10, wherein the anti-inflammatory agent is selected from the group consisting of a steroid and a non-steroidal anti-inflammatory (NSAID) agent.

13. The method of claim 4, wherein the method includes reducing energy input into the manufacturing process.

14. The method of claim 13, wherein energy input is reduced at a step selected from the group consisting of mixing, transferring, filtering and filling.

15. The method of claim 13, wherein the formation of non-equilibrium aggregates of the agent is reduced or eliminated.

16. The method of claim 4, wherein the composition is formulated for direct administration.

17. The method of claim 4, wherein manufactured composition is a solution, an emulsion or a suspension.

18. The method of claim 4, further comprising the step of assessing the surface tension of the manufactured composition.

19. The method of claim 13, wherein energy input is reduced at a step of filtering by reducing the pressure differential across the filter or reducing the turbulence during filtration.

20. A method of manufacture, comprising:
formulating at a manufacturing scale a pharmaceutical composition that has a predetermined surface tension, wherein:
the composition comprises tobramycin and a surfactant; and
the concentration of surfactant is near the critical micelle concentration, whereby the manufactured composition has a predetermined surface tension between about 30 to about 50 dynes/cm.

21. The method of claim 20, wherein the manufacturing process includes a reduced pressure differential during filtration or during filling.

22. A composition produced by the method of claim 4.

23. The composition of claim 22, wherein the volume manufactured is at least ten liters.

24. A manufactured composition, comprising:
a pharmaceutical agent; and
a surfactant at a concentration near or at the critical micelle concentration, whereby the manufactured composition has a predetermined surface tension; and
the volume of the manufactured composition is ten liters or greater.

25. The composition of claim 24, wherein the surfactant is a non-ionic surfactant.

26. The composition of claim 25, wherein the non-ionic surfactant is polysorbate-20.

27. The composition of claim 24, wherein the agent is an anti-infective agent or an anti-inflammatory agent.

28. The composition of claim 24, wherein the predetermined surface tension is between 30-50 dynes/cm.

29. The composition of claim 24, wherein the predetermined surface tension is between 35-45 dynes/cm.

30. An article of manufacture, comprising:
a pharmaceutical composition in a sterile container; wherein:
the composition comprises a pharmaceutical agent and a surfactant;
the surfactant is at a concentration near or at the critical micelle concentration;
the composition is sterile and has a predetermined surface tension; and
the composition is packaged aseptically.

31. The article of manufacture of claim 30, wherein the composition is packaged hermetically.

32. The article of manufacture of claim 30, wherein the composition is packaged in a container selected from the group consisting of a heat-sealed container, a vacuum-sealed container and a safety-sealed container.

33. The article of manufacture of claim 30, wherein the composition is packaged in a vial or ampoule.

34. The article of manufacture of claim 30, wherein the composition is packaged in a plastic or glass container.

35. The article of manufacture of claim 30, wherein the predetermined surface tension is between 30-50 dynes/cm.

36. The article of manufacture of claim 30, wherein the container is a unit dose or multi-dose container.

37. The article of manufacture of claim 30, wherein the composition is for direct administration.

38. The article of manufacture of claim 30, wherein the agent is an anti-infective or an anti-inflammatory agent.

39. The article of manufacture of claim 30, wherein the agent is an agent effective for the treatment of sinusitis.

40. The article of manufacture of claim 30, wherein the surfactant is a non-ionic surfactant.

41. The article of manufacture of claim 30, wherein the surfactant is not benzalkonium chloride.

42. A pharmaceutical composition, comprising:
tobramycin at a concentration of between about or at 15 mg/ml to about or at 60 mg/ml;
polysorbate-20 at a concentration of 0.125 mg/ml or at a concentration between 0.009% and 0.015% by weight;
sodium chloride at a concentration between 2.5 mg/ml and 4.3 mg/ml;
a volume greater than about or at ten liters; and
a predetermined surface tension between about or at 30 dynes/cm to about or at 50 dynes/cm.

43. An article of manufacture of claim 31, wherein the composition comprises:
tobramycin at a concentration of between about or at 15 mg/ml to about or at 60 mg/ml;
polysorbate-20 at a concentration of 0.125 mg/ml or at a concentration between 0.009% and 0.015% by weight;
sodium chloride at a concentration between 2.5 mg/ml and 4.3 mg/ml; and
the composition has a surface tension between about or at 30 dynes/cm to about or at 50 dynes/cm.

44. A system of manufacture for a formulation of at least 10 liters of a pharmaceutical composition, comprising:
an agent and a surfactant, wherein the amount of surfactant is near the critical micelle concentration; and
a filtration apparatus containing a filter, wherein the filter is for sterilization of the pharmaceutical composition and of a configuration that produces low turbulent flow, whereby the resulting composition is produced at a stable predetermined surface tension.

45. The system of claim 44, wherein the surfactant is a polysorbate.

46. The system of claim 44, wherein the filter has a pore size and/or pore distribution that permits or promotes laminar flow.

47. The system of claim 44, further comprising an automated apparatus for aseptically filling and hermetically sealing containers.

48. The system of claim 44, further comprising a computer for directing automated operation of the system.

49. A method of treating chronic sinusitis, comprising:
unsealing the container in the article of manufacture of claim 30;
transferring the composition contained in the container into a nebulizing device; and
nebulizing the composition, wherein the composition comprises an agent effective for treating sinusitis.

50. The method of claim 49, wherein the agent is an anti-infective or an anti-inflammatory.

51. The method of claim 49, wherein the agent is tobramycin.

52. The method of claim 49, wherein the composition comprises a non-ionic surfactant.

53. The method of claim 49, wherein the composition has a predetermined surface tension between about or at 30 dynes/cm to about or at 50 dynes/cm.

54. A method of manufacturing a composition with a predetermined surface tension, comprising:
dispersing or dissolving an active agent in a liquid in a formulation tank;
adding a surfactant to the active agent composition, wherein the final concentration of the surfactant in the composition is near the critical micelle concentration;
mixing the composition;
optionally adjusting the pH of the composition;
optionally adding other components to the composition;
transferring the composition to a filtration unit and filtering the composition under conditions that reduce or inhibit formation of non-equilibrium aggregates; and
packaging the filtered composition.

55. The method of claim 54, wherein the concentration of the surfactant is below the critical micelle concentration.

56. The method of claim 54, wherein the concentration of the surfactant is within 2 fold or 3 fold below or above the critical micelle concentration.

57. The method of claim 54, wherein the predetermined surface tension is between 30-50 dynes/cm.

58. The method of claim 57, wherein the predetermined surface tension is between 35-45 dynes/cm.

59. The method of claim 54, wherein the agent is selected from the group consisting of an anti-infective agent, an anti-inflammatory agent, an antihistamine, an antileukotriene, a decongestant, an antiviral agent, and an anesthetic.

60. The method of claim 59, wherein the anti-infective is selected from the group consisting of an aminoglycoside, a macrolide, a penicillin, a quinolone, a cephalosporin, an amphotericin B, a polyene, a pyrimidine analog and an azole agent.

61. The method of claim 59, wherein the anti-inflammatory agent is selected from the group consisting of a steroid and a non-steroidal anti-inflammatory (NSAID) agent.

62. The method of claim 54, wherein the method includes reducing energy input into the manufacturing process.

63. The method of claim 62, wherein energy input is reduced at a step selected from the group consisting of mixing, transferring, filtering and filling.

64. The method of claim 54, wherein the filter is a sterilizing filter having a pore size selected from the group consisting of 0.22, 0.2 or 0.1 micron.

65. The method of claim 64, wherein the sterile composition is aseptically packaged.

66. The method of claim 54, wherein the composition is packaged hermetically.

67. The method of claim 54, wherein the composition is packaged in a container selected from the group consisting of a heat-sealed container, a vacuum-sealed container or a safety-sealed container.

68. The method of claim 54, wherein the composition is packaged in a vial or ampoule.

69. The method of claim 54, wherein the agent is an agent effective for the treatment of sinusitis.

70. The method of claim 54, wherein the surfactant is a non-ionic surfactant.

71. The method of claim 54, wherein the agent is tobramycin and the surfactant is polysorbate-20.

72. The method of claim 71, wherein
the tobramycin is at a concentration of between about or at 15 mg/ml to about or at 60 mg/ml;
the polysorbate-20 is at a concentration of 0.125 mg/ml or at a concentration between 0.009% and 0.015% by weight; and
the composition has a predetermined surface tension between about or at 30 dynes/cm to about or at 50 dynes/cm.
